(19) United States
(12) Patent Application Publication
(10) Pub. No.: US 2024/0278249 A1
(43) Pub. Date: Aug. 22, 2024

(54) SURFACE-BASED DETECTION OF NUCLEIC ACID IN A CONVECTION FLOW FLUIDIC DEVICE

(71) Applicant: William Marsh Rice University, Houston, TX (US)

(72) Inventors: Dmitriy KHODAKOV, Houston, TX (US); David ZHANG, Houston, TX (US)

(73) Assignee: William Marsh Rice University, Houston, TX (US)

(21) Appl. No.: 18/449,422

(22) Filed: Aug. 14, 2023

Related U.S. Application Data
(62) Division of application No. 16/089,496, filed on Sep. 28, 2018, now abandoned.
(60) Provisional application No. 62/314,909, filed on Mar. 29, 2016.

(51) Int. Cl. B01L 7/00; B01L 3/00; C12Q 1/6844

(52) U.S. Cl. CPC B01L 7/52; B01L 3/5027; C12Q 1/6844; B01L 2200/0663; B01L 2300/0819; B01L 2300/1805; C12Q 2565/513; C12Q 2565/629

(57) ABSTRACT

The present disclosure provides methods, composition and devices for performing convection-based PCR and non-enzymatic amplification of nucleic acid sequences. Techniques and reagents employed in these methods include toehold probes, strand displacement reactions, Rayleigh-Benard convection, temperature gradients, multiplexed amplification, multiplexed detection, and DNA functionalization, in open and closed systems, for use in nucleic tests and assays.

### PRIORITY CLAIM

This application is a continuation of U.S. application Ser. No. 16/089,496, filed Sep. 28, 2018, which is a national phase application under 35 U.S.C. § 371 of International Application No. PCT/US2017/024530, filed Mar. 28, 2017, which claims benefit of priority to U.S. Provisional Application No. 62/314,909, filed Mar. 29, 2016, the entire contents of each of which are hereby incorporated by reference.

### SEQUENCE LISTING

This application contains a Sequence Listing XML, which has been submitted electronically and is hereby incorporated by reference in its entirety. Said Sequence Listing XML, created on Aug. 14, 2023, is named RICEP0022USC1.xml and is 86,220 bytes in size.

### FIELD

The disclosure describes novel reagents, instruments, and methods for detection and quantitation of specific nucleic acid sequences for scientific and clinical research and diagnostics applications.

### BACKGROUND

Most commercial nucleic acid (NA) assays require the use of enzymes for molecular or signal amplification. Enzymes such as DNA polymerase have been optimized to be fast and specific. Reconstitution of lyophilized enzymes in resource-limited conditions reduces the need for a cold chain. Isothermal nucleic acid amplification assays such as NEAR, LAMP and NASBA enable DNA/RNA profiling without complex temperature cycling equipment. Despite these many advances, existing nucleic acid detection technologies still face challenges for rapid PoC (point of care) detection of pathogen biomarkers, because it is difficult to design/evolve enzymes that simultaneously capture all desirable properties (e.g., fast, high fidelity, and robust to chemicals/inhibitors).

A number of existing nucleic acid analysis technologies are enzyme-free, including microarrays, fluorescence in situ hybridization (FISH), branched DNA dendrimers (Panomics), and fluorescent barcoding (Nanostring). In these approaches, the DNA or RNA target molecules stoichiometrically recruit or are converted into a limited number of fluorescent groups. This is unlike PCR where even a single nucleic acid molecule is amplified endlessly to produce an arbitrarily high number of amplicon molecules. Consequently, expensive and bulky equipment is needed for these approaches to achieve the molecular sensitivity needed to detect and analyze the small amounts of DNA or RNA target present in biological samples, restricting their use for PoC applications and in the limited resources conditions.

Another group of nucleic acid identification techniques employs solution-based enzyme-free DNA amplification approaches. Here a single-stranded DNA target molecule can catalytically release DNA oligonucleotides with identical sequence to the target from the preassembled DNA detection complexes in unlimited manner. Clinically relevant limits of detection have yet to be demonstrated for this family of approaches. In these systems, there is false positive amplification due to DNA “breathing” events that result in release of amplicon molecules in the absence of the detection of a target sequence.

### SUMMARY

Thus, in accordance with the present disclosure, there is provided a device comprising a surface having a plurality oligonucleotide complexes, wherein said oligonucleotide complexes each comprise:
a first oligonucleotide comprising a first DNA sequence and a linking moiety for irreversibly linking the first oligonucleotide to the surface, and
a second oligonucleotide comprising a second DNA sequence and a third DNA sequence, wherein the second DNA sequence is complementary to the first DNA sequence and is hybridized thereto, wherein said second DNA oligonucleotide does not comprise a fluorescent moiety and not irreversibly linked to the surface.
   Each of said second DNA sequences may be identical, or may not be identical. The plurality of said oligonucleotide complexes may be located in spatially discrete regions on said surface. The first oligonucleotides may comprise a fluorescent moiety, and each of said second oligonucleotides may comprise a fluorescence quencher. Each of said spatially discrete regions may further comprise a third oligonucleotide comprising a fourth DNA sequence and a fifth DNA sequence, wherein the fourth DNA sequence is complementary to the third DNA sequence. The third oligonucleotides may each comprise a fluorescence quencher moiety. The linking moiety may comprise an alkyne group including a strained alkyne or an azide group.

Each of the first DNA sequences may have a length of between about 15 and about 80 nucleotides. Each of the third DNA sequences may have a length of between about 5 and about 80 nucleotides, or a length of between about 15 and about 80 nucleotides. Each of the fifth DNA sequences may have a length of between about 5 and about 20 nucleotides. Each of the fourth DNA sequences may have a length of between about 5 and about 80 nucleotides.

In another embodiment, there is provided a fluidic reaction chamber comprising:
a first surface,
a second surface that does not contact the first surface, wherein said first and second surfaces face each other,
a material contacting the first surface and the second surface and that forms an outer boundary of said reaction chamber, and
a material contacting the first surface and the second surface and that forms and inner boundary of said reaction chamber,
wherein the first surface comprises a plurality of oligonucleotide complexes, wherein said oligonucleotide complexes each comprise:
   a first oligonucleotide comprising a first DNA sequence and a linking moiety for irreversibly linking the first oligonucleotide to the surface, and
   a second oligonucleotide comprising a second DNA sequence and a third DNA sequence, wherein the second DNA sequence is complementary to the first DNA sequence and is hybridized thereto, wherein said second DNA oligonucleotide is not irreversibly linked to the first surface, and optionally does not comprise a fluorescent moiety.
      Each of said second DNA sequences may be identical or may be not identical. Each of said plurality of said oligonucleotide complexes may be located in spatially discrete regions on said surface. Each of said first oligonucleotides may comprise a fluorescent moiety. Each of said second oligonucleotides may comprise a fluorescence quencher moiety. Each of said spatially discrete regions further may comprise a third oligonucleotide comprising a fourth DNA sequence and a fifth DNA sequence, wherein the fourth DNA sequence is complementary to the third DNA sequence. The third oligonucleotides may each comprise a fluorescence quencher moiety. The linking moiety may comprise an alkyne group including a strained alkyne or an azide group. The fluidic reaction chamber may have a first port to deliver the sample, and optionally a second another port to allow air/fluid exit when sample is introduced into the first port.

Each of the first DNA sequences may have a length of between about 15 and about 80 nucleotides. Each of the third DNA sequences may have a length of between about 5 and about 80 nucleotides. Each of the third DNA sequences may have a length of between about 15 and about 80 nucleotides. Each of the fifth DNA sequences may have a length of between about 5 and about 20 nucleotides. Each of the fourth DNA sequences may have a length of between about 5 and about 80 nucleotides. The materials contacting first and second surfaces that form the inner and outer boundaries of the chamber may have thickness between 40 microns (40 μm) and 2 millimeters (2 mm).

The fluidic reaction chamber may be circular, oval, square, rectangular, triangular, hexagonal, octagonal, rhomboid or trapezoid, or annular as defined herein. The fluidic reaction chamber may not be at a uniform temperature, and the warmest region of the reaction chamber may be at least 10° C. higher than the coldest region of the reaction chamber. The coldest region of the reaction chamber may be between about 50° C. and about 75° C. The hottest region of the reaction chamber may be between about 80° C. and about 100° C. The fluidic reaction chamber may further comprise a fluid disposed within the fluidic reaction chamber, said fluid solution comprising a DNA polymerase, dNTPs, and PCR buffer.

In yet another embodiment, there is provided a method of amplifying a target nucleic acid comprising (a) providing a fluidic reaction chamber according to claim 16, wherein said fluidic reaction chamber is in operable relationship to a first and a second heat source, wherein said first and second heat sources are capable of applying differing first and a second heat levels to said annular chamber, wherein said first and second heat levels are not the same; (b) introducing into said fluidic reaction chamber a fluid comprising a target nucleic acid sequence, a DNA polymerase, dNTPs and a polymerase chain reaction (PCR) buffer; and (c) applying first and second heat levels to said fluidic reaction chamber. The method may further comprise detecting amplification of said target nucleic acid.

Each of said second DNA sequences may be identical or may not be identical. The plurality of said oligonucleotide complexes may be located in spatially discrete regions on said surface. Each of said first oligonucleotides may comprise a fluorescent moiety, and each of said second oligonucleotides may comprise a fluorescence quencher moiety. Each of said spatially discrete regions may further comprise a third oligonucleotide comprising a fourth DNA sequence and a fifth DNA sequence, wherein the fourth DNA sequence is complementary to the third DNA sequence. The third oligonucleotides may each comprise a fluorescence quencher moiety. The linking moiety may comprise an alkyne group including a strained alkyne or an azide group.

The first DNA sequences may have a length of between about 15 and about 80 nucleotides. Each of the third DNA sequences may have a length of between about 5 and about 80 nucleotides. Each of the third DNA sequences may have a length of between about 15 and about 80 nucleotides. Each of the fifth DNA sequences have a length of between about 5 and about 20 nucleotides. The fluidic reaction chamber may be circular, oval, square, triangular, rectangular, hexagonal, octagonal, rhomboid or trapezoid.

The fluidic reaction chamber may not at a uniform temperature, and the warmest region of the reaction chamber may be at least 10° C. higher than the coldest region of the reaction chamber. The coldest region of the reaction chamber may be between about 50° C. and about 75° C. The hottest region of the reaction chamber may be between about 80° C. and about 100° C.

In yet a further embodiment, there is provided a device comprising a first surface region and a second surface region,
the first surface region comprising
   a first oligonucleotide comprising a first DNA sequence and a linking moiety for irreversibly linking the first oligonucleotide to the first surface region, and
   a second oligonucleotide comprising a second DNA sequence and a third DNA sequence, wherein the second sequence is complementary to the first sequence, and
the second surface region comprising
   a third oligonucleotide comprising a fourth DNA sequence and a linking moiety for irreversibly linking the third oligonucleotide to the second surface region, and
      wherein the fourth sequence is complementary to the second sequence, the third sequence, or a combination of at least six continuous nucleotides of the second sequence and six continuous nucleotides of the third sequence,
         or a device comprising a first surface region and a second surface region,
the first surface region comprising
   a first oligonucleotide comprising a first DNA sequence and a linking moiety for irreversibly linking the first oligonucleotide to the first surface region, and
   a second oligonucleotide comprising a second DNA sequence and a third DNA sequence, wherein the second sequence is complementary to the first sequence, and
the second surface region comprising
   a third oligonucleotide comprising a fourth DNA sequence and a linking moiety for irreversibly linking the third oligonucleotide to the second surface region, and
   a fourth oligonucleotide comprising a fifth DNA sequence and a sixth DNA sequence, wherein the fifth sequence is complementary to the fourth sequence, and
      wherein the second sequence is complementary to the fifth sequence or is complementary to the sixth sequence.
         The first or second oligonucleotide may comprises a fluorescent moiety, and the first or second oligonucleotide may comprise a fluorescence quencher. The second DNA sequences may be identical or may not be identical. The plurality of said oligonucleotide complexes may be located in spatially discrete regions on said surface. The linking moiety may comprise an alkyne group including a strained alkyne or an azide group. The fluidic reaction chamber may have a first port to deliver the sample, and optionally a second another port to allow air/fluid exit when sample is introduced into the first port.

Each of the first DNA sequences may have a length of between about 15 and about 80 nucleotides. Each of the third DNA sequences may have a length of between about 5 and about 80 nucleotides. Each of the third DNA sequences may have a length of between about 15 and about 80 nucleotides. Each of the fifth DNA sequences may have a length of between about 5 and about 20 nucleotides. Each of the fourth DNA sequences may have a length of between about 5 and about 80 nucleotides. Each of the sixth DNA sequences may have a length of between 5 and 80 nucleotides.

In still a further embodiment, there is provided a fluidic reaction chamber comprising:
a first surface,
a second surface that does not contact the first surface, wherein said first and second surfaces face each other,
a material contacting the first surface and the second surface and that forms an outer boundary of said reaction chamber, and
a material contacting the first surface and the second surface and that forms and inner boundary of said reaction chamber, wherein
(a) the first surface comprises
   a first oligonucleotide comprising a first DNA sequence and a linking moiety for irreversibly linking the first oligonucleotide to the first surface region, and
   a second oligonucleotide comprising a second DNA sequence and a third DNA sequence, wherein the second sequence is complementary to the first sequence, and
   the second surface region comprises
   a third oligonucleotide comprising a fourth DNA sequence and a linking moiety for irreversibly linking the third oligonucleotide to the second surface region, and
      wherein the fourth sequence is complementary to the second sequence, the third sequence, or a combination of at least six continuous nucleotides of the second sequence and six continuous nucleotides of the third sequence; or
(b) the first surface region comprises
   a first oligonucleotide comprising a first DNA sequence and a linking moiety for irreversibly linking the first oligonucleotide to the first surface region, and
   a second oligonucleotide comprising a second DNA sequence and a third DNA sequence, wherein the second sequence is complementary to the first sequence, and
   the second surface region comprises
   a third oligonucleotide comprising a fourth DNA sequence and a linking moiety for irreversibly linking the third oligonucleotide to the second surface region, and
   a fourth oligonucleotide comprising a fifth DNA sequence and a sixth DNA sequence, wherein the fifth sequence is complementary to the fourth sequence, and
      wherein the second sequence is complementary to the fifth sequence or is complementary to the sixth sequence.
         The first or second oligonucleotide may comprise a fluorescent moiety, and the first or second oligonucleotide may comprises a fluorescence quencher. Each of said second DNA sequences may be identical or may not be identical. Each of said plurality of said oligonucleotide complexes may be located in spatially discrete regions on said surface. The linking moiety may comprises an alkyne group including a strained alkyne or an azide group. The fluidic reaction chamber may have a first port to deliver the sample, and optionally a second another port to allow air/fluid exit when sample is introduced into the first port.

Each of the first DNA sequences may have a length of between about 15 and about 80 nucleotides. Each of the third DNA sequences may have a length of between about 5 and about 80 nucleotides. Each of the third DNA sequences may have a length of between about 15 and about 80 nucleotides. Each of the fifth DNA sequences may have a length of between about 5 and about 20 nucleotides. Each of the fourth DNA sequences may have a length of between about 5 and about 80 nucleotides. Each of the sixth DNA sequences may have a length of between about 5 and about 80 nucleotides.

The fluidic reaction chamber may be circular, oval, square, triangular, rectangular, hexagonal, octagonal, rhomboid or trapezoid, or annular as defined herein. The fluidic reaction chamber may not be at a uniform temperature, and the warmest region of the reaction chamber may be at least 10° C. higher than the coldest region of the reaction chamber. The coldest region of the reaction chamber may be between about 10° C. and about 50° C. The hottest region of the reaction chamber may be between about 51° C. and about 100° C. The fluidic reaction chamber may further comprise a fluid disposed within the fluidic reaction chamber, said fluid comprising one or more oligonucleotides and hybridization buffer, and the fluid may further comprise a non-specific nucleic acid staining dye.

Yet an additional embodiment comprises a method of amplifying a target nucleic acid comprising (a) providing a fluidic reaction chamber according to claim 71, wherein said fluidic reaction chamber is in operable relationship to a first and a second heat source, wherein said first and second heat sources are capable of applying differing first and a second heat levels to said annular chamber, wherein said first and second heat levels are not the same; (b) introducing into said fluidic reaction chamber a fluid comprising a target nucleic acid sequence; and (c) applying first and second heat levels to said fluidic reaction chamber.

The method may further comprise detecting amplification of said target nucleic acid. The first or second oligonucleotide may comprise a fluorescent moiety, and the first or second oligonucleotide may comprise a fluorescence quencher. Each of said second DNA sequences may be identical or may not be identical. Each of said plurality of said oligonucleotide complexes are located in spatially discrete regions on said surface. The linking moiety may comprise an alkyne group including a strained alkyne or an azide group.

Each of the first DNA sequences may have a length of between about 15 and about 80 nucleotides. Each of the third DNA sequences may have a length of between about 5 and about 80 nucleotides. Each of the third DNA sequences may have a length of between about 15 and about 80 nucleotides. Each of the fifth DNA sequences may have a length of between about 5 and about 20 nucleotides. Each of the fourth DNA sequences may have a length of between about 5 and about 80 nucleotides. Each of the sixth DNA sequences may have a length of between about 5 and about 80 nucleotides.

The fluidic reaction chamber may be circular, oval, square, rectangular, triangular, hexagonal, octagonal, rhomboid or trapezoid, or annular as defined herein. The fluidic reaction chamber may not be at a uniform temperature, and the warmest region of the reaction chamber may be at least 10° C. higher than the coldest region of the reaction chamber. The coldest region of the reaction chamber may be between about 10° C. and about 50° C. The hottest region of the reaction chamber may be between about 51° C. and about 100° C. The fluid further comprises a non-specific nucleic acid staining dye.

Another embodiment comprises a system comprising a reaction chamber, comprising (a) a first region and second region, wherein a first oligonucleotide comprising a first nucleotide sequence is functionalized to the first surface region and a second oligonucleotide comprising a second nucleotide sequence is functionalized to the second surface region, and wherein the first nucleotide sequence and the second nucleotide sequence are not identical; (b) a buffer solution amenable for DNA hybridization at a non-uniform temperature, wherein the buffer solution contacts the first surface region and the second surface region; (c) a third oligonucleotide comprising a third nucleotide sequence, wherein the third oligonucleotide is hybridized to the first oligonucleotide; and (d) a first temperature zone and a second temperature zone, wherein the first temperature zone has a temperature at least 10° C. greater than a temperature of the second temperature zone.

The first surface region may be located on a first surface and the second surface region is located on the first surface. The first surface region may be located on a first surface and the second surface region is located on a second surface, wherein the first surface and the second surface are different surfaces. The first nucleotide sequence may comprise a first nucleotide region that is not complementary to the third nucleotide sequence. The third nucleotide sequence may comprise a second nucleotide region that is not complementary to first nucleotide sequence.

The buffer solution may comprise at least 60% by mass water and a cation at a concentration of at least 1 mM. The length of the first oligonucleotide and the length of the second oligonucleotide may be between 5 nucleotides and 20,000 nucleotides. The first oligonucleotide and the length of the second nucleotide may be between 5 nucleotides and 200 nucleotides. The length of the third oligonucleotide may be between 5 nucleotides and 20,000 nucleotides. The first oligonucleotide, the second oligonucleotide and the third oligonucleotide may be identical or differently and may comprise a nucleic acid selected from the group consisting of DNA, RNA, a nucleotide analog, and any combination thereof.

The nucleotide analog may be selected from the group consisting of LNA, PNA a morpholino-oligonucleotide, and any combination thereof. At least one of the first oligonucleotide, the second oligonucleotide and the third oligonucleotide may be functionalized with a chemical moiety, wherein the chemical moiety allows detection of oligonucleotides. The chemical moiety may be selected from the group consisting of TAMRA, ROX, HEX, an organic fluorophore, a quantum dot, a nanoparticle, methylene blue, an electrochemically active molecule, and any combination thereof.

The buffer solution may comprise a detectable molecule, wherein the detectable molecule exhibits a different unit signal when non-irreversibly bound to an oligonucleotide than when free in solution, such as detectable molecule selected from the group consisting of a SybrGreen dye, a Syto dye, and a EvaGreen dye. The first surface and the second surface may be identical or differently selected from the group consisting of glass, quartz, plastic, a polymer, metal, composite material, and surface self-assembled monolayers. The polymer may be PDMS.

The first surface region may comprise a temperature that is 10° C. below a maximum temperature of the buffer solution, and wherein the second surface region may comprise a temperature that is 10° C. below the maximum temperature of the buffer solution. The system may further comprise at least one heating/cooling element in contact with the first surface region and the second surface region. The at least one heating/cooling element may be selected from the group consisting of a hot plate, a heating fan, an IR-heater, and a water bath. The hot plate may be selected from the group consisting of a thermo-resistive heater and a Peltier element. The system may further comprise an enzyme that modifies nucleic acids in a template-directed manner, such as where the enzyme facilitates template-directed extension of a nucleic acid template.

A method for enzyme-free amplification and detection of a nucleic acid target, comprising (a) contacting a sample with a composition in a reaction chamber, wherein the composition comprises:
a buffer solution amenable for DNA hybridization;
a first surface region and a second surface region, wherein the buffer solution contacts the first surface region and the second surface region;
a first oligonucleotide comprising a first nucleotide sequence, wherein the first oligonucleotide is functionalized to the first surface region;
a second oligonucleotide comprising a second nucleotide sequence, wherein the second oligonucleotide is functionalized to the second surface region; and
a third oligonucleotide comprising a third nucleotide sequence, wherein the third oligonucleotide is hybridized to the first oligonucleotide,
wherein the first nucleotide sequence and the second nucleotide sequence are not identical, and wherein the third nucleotide comprises a first nucleotide region that is not complementary to the first nucleotide sequence;
(b) heating differentially a portion of the reaction chamber, wherein a maximum temperature of a region of the chamber is at least 10° C. higher than a minimum temperature of the chamber; and (c) detecting potential amplification.
   The reaction chamber may comprise at least one material selected from the group consisting of glass, quartz, plastic, a polymer, metal, and any combination thereof. The polymer may be PDMS. The maximum temperature of a region of the chamber may be between 60° C. and 100° C. The minimum temperature of the chamber may be between 20° C. and 60° C. The first surface region and the second surface region may be not heated to within 10° C. of the maximum temperature of a region of the chamber.

The composition may be localized in the reaction chamber, and wherein the first surface region is located on first surface and the second surface region is located the first surface. The composition may be localized in the reaction chamber, wherein the first surface region may be located on a first surface and the second surface region may be located on a second surface, and wherein the first surface and the second surface are different surfaces. Detecting potential amplification may further comprise optical detection of fluorescence changes through a detection device selected from the group consisting of a photodiode, a photomultiplier tube, a fluorescence microscope, a CCD camera, and any other optical detection device. Detecting potential amplification may further comprise electrochemical detection through an electrochemical potentiostat/galvanostat. Detecting potential amplification may further comprise measuring the mass of the first surface region using quartz crystal microbalance technique.

In still a further embodiment, there is provided a method for enzyme-dependent amplification and detection of a nucleic acid target, comprising (a) contacting a sample with a composition in a reaction chamber, wherein the composition comprises:
a buffer solution amenable for DNA hybridization;
a surface region, wherein the buffer solution contacts the surface region;
a first oligonucleotide comprising a first nucleotide sequence, wherein the first oligonucleotide is functionalized to the surface region;
a second oligonucleotide comprising a second nucleotide sequence; and an enzyme that modifies nucleic acids in a template-directed manner, wherein the second nucleotid sequence comprises a nucleotide region that is not complementary to the first nucleotide sequence;
(b) heating differentially a portion of the reaction chamber, wherein a maximum temperature of a region of the chamber is at least 10° C. higher than a minimum temperature of the chamber; and (c) detecting potential amplification.
   The first oligonucleotide m a y comprise a second nucleotide region that is not complementary to the second nucleotide sequence. The enzyme may be selected from the group consisting of a DNA polymerase, a RNA polymerase, a reverse transcriptase, an endonuclease, and an exonuclease. The reaction chamber may comprise at least one material selected from the group consisting of glass, quartz, plastic, a polymer, metal, and any combination thereof. The polymer may be PDMS.

The maximum temperature of a region of the chamber may be between 80° C. and 100° C. The minimum temperature of the chamber may be between 20° C. and 80° C. The surface region may not be heated to within 10° C. of the maximum temperature of a region of the chamber. Detecting potential amplification may further comprise optical detection of fluorescence changes through a detection device selected from the group consisting of a photodiode, a photomultiplier tube, a fluorescence microscope, a CCD camera, and any other optical detection device. Detecting potential amplification may further comprise electrochemical detection through an electrochemical potentiostat/galvanostat. Detecting potential amplification may further comprise measuring the mass of the first surface region using quartz crystal microbalance technique.

In one embodiment of the present disclosure, surface oligonucleotide functionalization can be performed by various chemical and physical methods including but not limited to covalent immobilization, electrostatic interaction or non-covalent immobilization such as biotin-avidin (or their analogs).

In one embodiment of the present disclosure, the detection target molecule may be single-stranded DNA, double-stranded DNA, RNA or their mixtures.

In one embodiment of the present disclosure, amplicon detection and reaction monitoring is through methods including but not limited to: fluorescence (including surface plasmon resonance, SPR), UV absorbance, electrochemical detection (including pH change and charge transfer), quartz crystal microbalance (QCM) In one embodiment of the present disclosure, the proposed approaches can be used for distinguishing nucleic acid sequence variants, including single nucleotide variants (SNVs) that may be indicative of drug resistance or disease prognosis.

In one embodiment of the present disclosure, the proposed approaches can be used for quantitation of one, several, or many target molecules in specific biological samples.

As used herein, the term “annular” may be used to reference the shape of the chamber, as discussed above, and may have its normal meaning of round, oval or discoid. However, annular may also be interpreted in this context to have other regular or irregular shapes so long as the chamber constitutes a continuous circuit with no end and no beginning.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

Throughout this application, the term “irreversible linking” is used to indicate a chemical interaction that is stable under usual circumstances of the intended application. Irreversible linking in some embodiments can refer to covalent attachment such as by azide-alkyne click chemistry, and in other embodiments can refer to biotin-avidin interactions or other non-covalent long-lived interactions.

Throughout this application, the term “PCR buffer” is used to indicate an aqueous solution with salinity and chemical composition compatible with DNA amplification by a DNA polymerase via the polymerase chain reaction (PCR). The buffer may be used in conjunction with the DNA polymerase itself, primers and/or dNTPS.

Throughput this application, the term “hybridization buffer” is used to indicate an aqueous solution with salinity and chemical composition compatible with DNA hybridization and formation of stable DNA duplexes by complementary DNA oligonucleotides. All PCR buffers can be considered hybridization buffers, but not vice versa.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a schematic of an embodiment of the fluidic chip vertically mounted on heaters at differential temperatures. The fluidic chip comprises a reaction chamber with a surface for functionalizing DNA oligonucleotides. In some embodiments, this chip is used for performing multiplex PCR-based detection of nucleic acids.

FIG. 2 shows an embodiment of the system, in which the fluidic chip is mounted vertically on the heaters at differential temperatures. A horizontally mounted light source excites fluorescent moieties on DNA functionalized to the chip; fluorescence signal is quantitated via the shown detector.

FIG. 3 shows camera pictures of the fluidic chip (left), the chip mounted on heaters (middle) and the chip with rectangular-shaped chambers mounted on the heaters (right).

FIG. 4 shows an example of the chemical approach for functionalization of the glass slide with DNA oligonucleotide to form part of the detection probe. The process is a two-step reaction of the PDITC (p-phenylene-diisothiocyanate) activated glass with an azido-PEG-amine (11-azido-3,6,9-trioxaundecan-1-amine) followed by a conjugation with an alkyne-functionalized oligonucleotide via copper-catalyzed alkyne-azide cyclo-addition that results in an oligonucleotide attachment through a hydrophilic PEG linker. Hydrophilicity of the functionalized surface prevents non-specific absorption of the reaction components such as target DNA, primers and enzymes. Copper-free reaction of the azide-functionalized surface with oligonucleotides modified with strained cycloalkynes also results in stable and highly specific covalent attachment of the oligonucleotides to the surface.

FIG. 5 shows an example of the functionalized probe structure. The oligonucleotide functionalized to the surface is labeled with a fluorophore and the oligonucleotide hybridized to the surface-functionalized probe is labeled with a quencher. When a detection target displaces the quencher-labeled oligonucleotide from the surface, the fluorescence intensity of the surface increases. Bottom panels show fluorescence microscopy images before and after target introduction; the spot diameter here is 1 mm. Oligonucleotides were as follows:

Alk-TMR-1
(SEQ ID NO: 1)
/5Hexynyl/tttt/i6-TAMN/tggatgctgaatacttgtgataa
taca
32-RQ
(SEQ ID NO: 2)
ccgtagaggtgtattatcacaagtattcagcatcca/3IAbRQSp/
54
(SEQ ID NO: 3)
tggatgctgaatacttgtgataatacacctctacgg

FIG. 6 shows a schematic for polymerase chain reaction (PCR) amplification of a genomic DNA (gDNA) template within the fluidic chip when convection flow is applied; sequence (10) indicates forward primer and sequence (11) indicates reverse primer. The double-stranded amplicon, comprising domains 10-15-16-17 in the forward strand and domains 11-12-13-14 in the reverse strand is denatured in the hot 95° C. zone, and then is carried by convection to the 60° C. zone, where it can displace a quencher-labeled oligonucleotide to generate increased fluorescence in the corresponding spot.

FIG. 7 shows results of PCR amplification within the convection chip. The left panel shows an agarose gel electrophoresis of amplification products. Lane 1 shows the amplification product of 10 ng of NA18562 gDNA template amplified for 30 minutes in the convection chip. Primer concentrations are 600 nM for the forward primer and 200 nM reverse primers. Lane 2 shows a negative control with primers, polymerase, dNTPs, and probe spot, but no gDNA input. The ladder is a 50 bp ladder (New England Biolabs) as a reference. The right panel shows fluorescence time course of the spot intensity through an amplification reaction. The probes and primers for this experiment were designed for target rs7517833 (see FIGS. 20A-20C).

FIG. 8 demonstrates convection flow in the fluidic chip. The top left panel shows a fluorescence image of fluorescent tracking beads in the absence of a temperature gradient across the chip (60° C. for both heaters). The top right panel shows a time-lapse (2 second) fluorescence image of the fluorescence tracking beads when a temperature gradient is applied (95° C. for left heater and 60° C. for right heater). The bottom panel summarizes observed mean convection flow velocity based on chamber thickness.

FIG. 9 shows a schematic for an array-based readout of multiple amplicons within the fluidic chip. The right panel shows a fluorescence image of the chip with 24 printed spots. Spots marked M are positive control spots lacking quencher-labeled oligonucleotides. Other spots each are specific to a particular amplicon sequence.

FIG. 10 shows fluorescence images of the probe array area of the fluidic chip before and after convection PCR. Primers that generate amplicons corresponding to the probes at spots 2, 3, and 4 were introduced (600 nM each forward primer, 200 nM each reverse primer), along with 10 ng of gDNA template. High fluorescence of spot 14 is unintentional and may have resulted from poorly functionalized or hybridized DNA probe molecules.

FIG. 11 shows time-course fluorescence for 9-plex PCR amplification in the convection fluidic chip. Each spot's fluorescence intensity was individually quantitated and normalized based on background fluorescence and the fluorescent intensity of the marker spots M. Each forward primer concentration is 200 nM and each reverse primer concentration is 100 nM, and gDNA input is 10 ng.

FIGS. 12A-D show 3-plex PCR amplification in the convection fluidic chip (FIG. 12A) corresponding to primers for human DNA (FIG. 12B), mouse DNA (FIG. 12C), and rat DNA (FIG. 12D). Here, all spots in a row have the same sequence identity and report on the same amplicon. The top row (C in FIG. 12A) are positive control probes. In the reaction chamber was 600 nM each forward primer, 200 nM each reverse primer, and 10 ng of gDNA template. Sequences used in the experiment were as follows:

### Primers

h_ppia_fp
(SEQ ID NO: 4)
gttaacagattggaggtagtagcatttt
h_ppia_rp
(SEQ ID NO: 5)
tctatcaccaccccccaact
r_b2m_fp
(SEQ ID NO: 6)
caggtattttggggtatgattatggtt
r_b2m_rp
(SEQ ID NO: 7)
ccaacagaatttaccaggaaacaca
m_gadph_fp
(SEQ ID NO: 8)
caatacggccaaatctgaaagacaa
m_gadph_rp
(SEQ ID NO: 9)
ctgcaggttctccacacctat

### Arms

h_ppia_arm
(SEQ ID NO: 10)
agcagtgcttgctgttccttagaattttgccttgtgcgatgctgaata
cttgtgataatacacctctacgggtcagg
r_b2m_arm
(SEQ ID NO: 11)
ctggttcttactgcagggcgtgggaggagcgcgatgctgaatacttgt
gataatacacctctacgggtcagg
m_gadph_arm
(SEQ ID NO: 12)
gatagcctggggctcactacagacccatgagggcgatgctgaatactt
gtgataatacacctctacgggtcagg

### Quenchers

h_ppia_q
(SEQ ID NO: 13)
/5IAbRQ/acaaggcaaaattctaaggaacagcaagcactgctgcacg
atcaggggt
r_b2m_q
(SEQ ID NO: 14)
/gctcctcccacgccctgcagtaagaaccagaccccagcctttacac
m_gadph_q
(SEQ ID NO: 15)
/5IAbRQ/cctcatgggtctgtagtgagccccaggctatctcatgttc
ttcagagtgga

### Anchor

(SEQ ID NO: 16)
/DBCO/tttttcctgacccgtagaggtgtattatcacaagtattcagc
atcgc/ATTO-550/

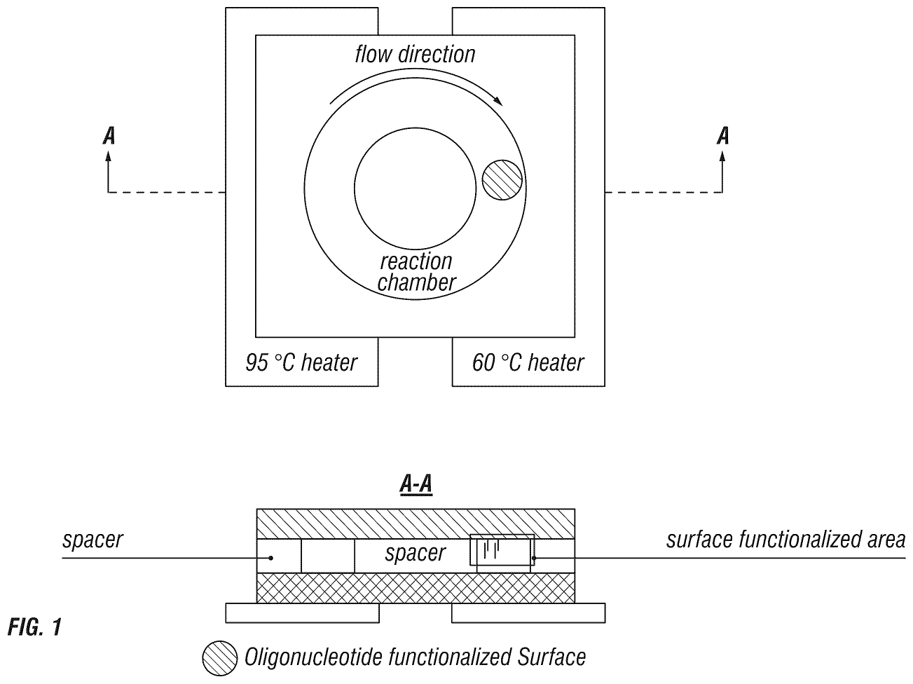

FIG. 13A shows a schematic of the reaction chamber with two surface regions, each functionalized with different DNA oligonucleotide reagents. Unlike in FIG. 1, the oligonucleotide reagents are not independent in sequence, but are rather rationally designed for enzyme-free amplification. FIG. 13B shows two possible embodiments of the two surface regions: either they are on different surfaces, or on the same surface but distally located to prevent direct interaction.

FIGS. 14A-B show the mechanism for linear amplification of a target nucleic acid sequence bearing a sixth sequence (6) and a seventh sequence (7). The target nucleic acid sequence catalytically transfers multiple oligonucleotides bearing the second sequence (2) and the third sequence (3) from surface region 1 to surface region 2. Spontaneous dissociation of the double-stranded DNA molecule (23:67) in the hot zone is critical to allow rapid turnover. FIG. 14C shows the net reaction of the process described in FIG. 14A, as well as a fluorescent labeling strategy to allow real-time readout. FIG. 14D shows an alternative implementation with flipped 5′/3′ orientation (the half arrow-head denotes 3′ end, as custom in literature).

FIGS. 15A-B show the mechanism for a control experiment in which the target nucleic acid sequence induces a stoichiometric rearrangement of surface-bound oligonucleotides.

FIG. 16 shows time-course fluorescence of surface region 1 when various concentrations of target nucleic acid are introduced. The fluorescence in the linear amplification chip decreases more quickly than in the stoichiometric conversion chip, supporting the mechanism of enzyme-free DNA amplification.

FIGS. 17A-B shows the mechanism for exponential amplification of a target nucleic acid sequence.

FIG. 18 shows a visual representation of reporting enzyme-free amplification through the use of an intercalating dye, such as SybrGreen or EvaGreen. The fluorescence intensity of surface region 1 will decrease through the course of the reaction, and the fluorescence intensity of surface region 2 will increase.

FIG. 19 shows a visual representation of reporting enzyme-free amplification through the use of a fluorophore-functionalized oligonucleotides. The fluorescence intensity of surface region 1 will increase through the course of the reaction, and the fluorescence intensity of surface region 2 will remain dark through the course of the reaction.

FIGS. 20A-C show the list of primers (FIG. 20A), arms (FIG. 20B), and quenchers and anchor (FIG. 20C) used for PCR amplification/detection.

### DETAILED DESCRIPTION

Here, the inventors present devices, systems, and methods for DNA amplification assay. The disclosure employs solid-phase separation of reagents to prevent unintended molecular events resulting in false positives, and uses convection flow circulation to enable spontaneous dissociation of double-stranded amplicons. Three related prior art technologies and their limitations compared to the present invention are described below.

### 1. CONVECTION FLOW PCR

Liquid, when held at a non-uniform temperature and confined in a volume, will circulate via a process known as Rayleigh-Benard convection flow [¹]. Rayleigh-Benard convection has been used for molecular diagnostics to generate low-cost devices for providing the necessary temperature cycling for PCR (convection flow PCR, cf-PCR) [², U.S. Pat. No. 6,586,233 B2, U.S. Pat. No. 8,735,103 B2, U.S. Pat. No. 8,187,813 B2]. cf-PCR requires only a static temperature gradient maintained with a high of around 95° C. and a low of around 60° C. (annealing/extension temperature), eliminating the need for high energy consumption thermal cycling instruments.

cf-PCR has been demonstrated for both single-plex [³⁻⁵, U.S. Pat. No. 8,187,813 B2] and multiplex detection of specific DNA sequences [⁶]; the multiplex approach utilized end-point electrophoretic results examination. Because cf-PCR lacks the temperature uniformity of traditional qPCR assays, cf-PCR struggles in applications requiring high sequence selectivity, such as applications for detection or profiling of single nucleotide variants (SNV), therefore no SNV specific cf-PCR has yet been shown. Real-time detection of the cf-PCR has been shown solely in solution phase employing unspecific fluorescent dye (SYBR Green I) detection method [⁷]. This approach restricts the cf-PCR from being used in multiplex settings. Likewise, application of sequence specific real-time detection methods such as 5′-nuclease assay chemistry or hybridization probes would allow detecting not more than 5-6 targets simultaneously because of fluorophore spectral overlap. The present disclosure is differentiated from cf-PCR in that the present disclosure offers spatially resolved multiplexed readout without requiring an open-tube step for subsequent analysis. Additionally, in the enzyme-free embodiment of the disclosure, no enzyme is required for amplification.

### 2. MICROARRAYS

Microarray technology is one of the main techniques for multiplexed screening of biological samples. Multiple probe sequences are functionalized to a surface, and the fluorescent signal of a particular spot is taken as the quantitative readout of the corresponding sequence. The technology has been successfully demonstrated for detecting of various types of biological analytes such as DNA, RNA, proteins, carbohydrates and cells [⁸⁻¹²]. Application of the microarray technology has found the most extensive use in the field of nucleic acid testing. Microarray technology has shown application of NA microarrays for whole genome hybridization, de novo sequencing, re-sequencing, comparative genomics, transcriptome hybridization or identification of single nucleotide variations [¹³⁻¹⁵]. All aforementioned NA applications require large amounts of NA targets for hybridization, consequently microarrays are typically used as a final readout on PCR amplification products. Microarray readouts are typically slow, requiring overnight hybridization, and also risks amplicon contamination due to the open-tube process.

### 3. TOEHOLD PROBES AND ENZYME-FREE AMPLIFICATION

Toehold-mediated strand displacement reaction [¹⁹⁻²¹] is a process of competitive hybridization that occurs in the absence of enzymes, and is relevant to the present disclosure. Using toehold-mediated strand displacement, enzyme-free amplification of DNA and RNA analyte sequences in homogeneous solutions has been demonstrated [²²⁻²⁷] (U.S. Pat. No. 8,043,810 B2, U.S. Pat. No. 8,110,353 B2). The enzyme-free amplification embodiment of the disclosure is different in that thermal convection flow is used to spontaneously dissociate double-stranded amplicons, and surface-functionalization is used to sequester reactive reagents from one another to reduce false positives. Toehold-mediated strand displacement has been applied to surface functionalized DNA oligonucleotides (U.S. Pat. No. 8,630,809 B2) for stoichiometric conversion of target analyte sequences to other sequences. The present disclosure differs in providing amplification of the detection target.

### 4. APPLICANTS' TECHNOLOGY

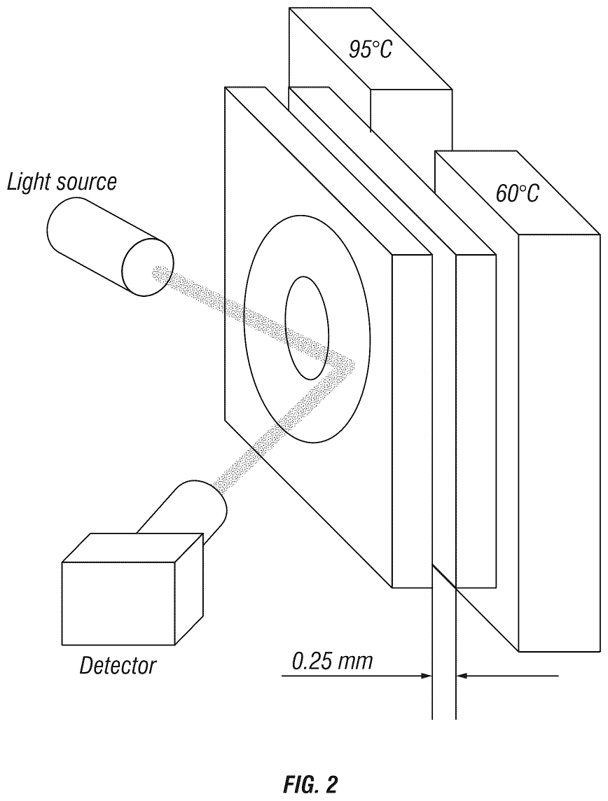

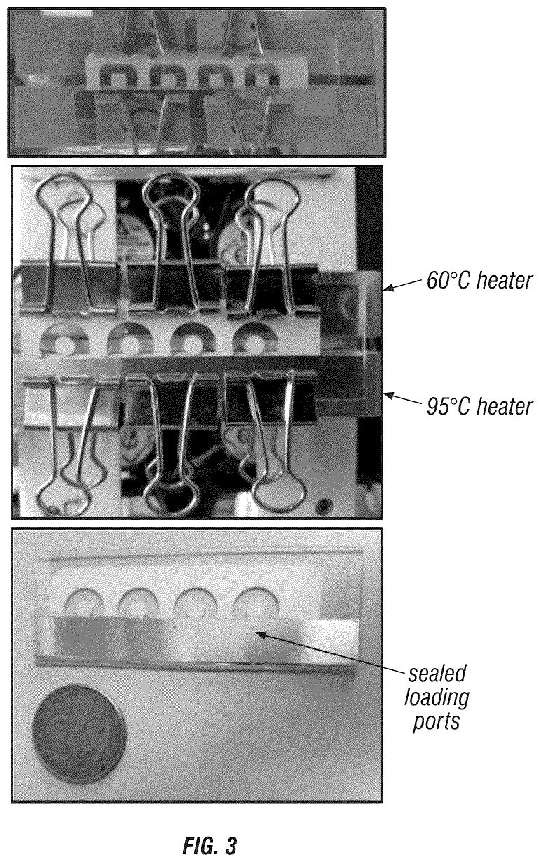

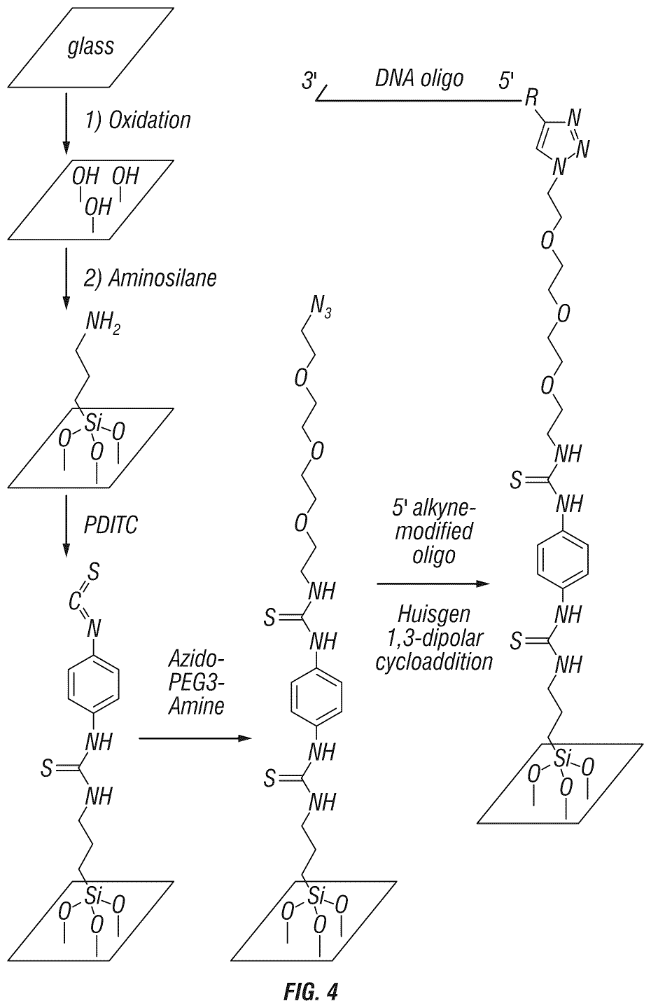

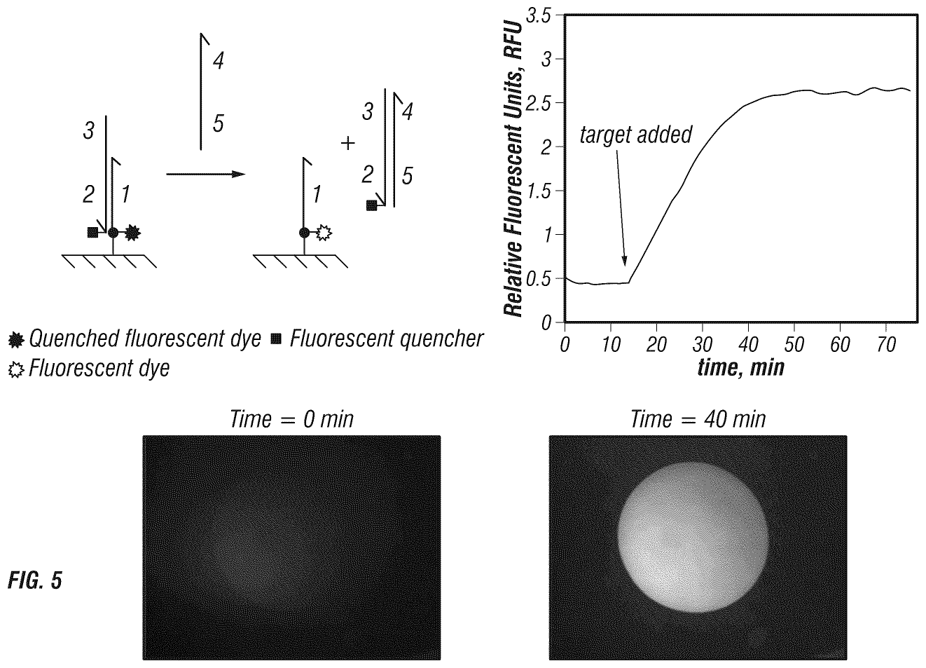

This disclosure describes reagents and devices for amplification and detection of specific nucleic acid target sequences. The disclosure utilizes solid-phase functionalization and sequestering of oligonucleotide reagents, in order to prevent unintended molecular events that result in false positives, and application of Rayleigh-Benard thermal convection flow for target regeneration and facilitating DNA surface hybridization kinetics (more efficient mixing of the reaction mixture). The Rayleigh-Benard convection flow regime can be realized by placing a reaction chamber, which consists of two 1 mm thick white-water glass microscope slides separated by double-sided sticky tape as a spacer with thickness of 250 μm, between two differentially-controlled hot plates (FIGS. 1-3) tilted at the defined angle. The shape of the spacer determines the shape of the reaction chamber, and can be modified to alter convection flow speed and trajectory. Glass is selected as the chip material because glass facilitates maintenance of a uniform temperature gradient across the chamber and allows surface functionalization with synthetic oligonucleotides.

The hot plates are set to maintain two different temperatures (cold heater and hot heater, respectively), which cause a temperature gradient across the reaction chamber filled with a liquid reaction mixture. Liquid residing near the hot part of the chamber has a higher temperature and, therefore, is less dense than the liquid residing in the part of the chamber with lower temperature. Such distribution of liquid densities in confined volume results in a difference between buoyancy and gravity forces (near the hot and cold heaters, respectively) that in turn results in organization of circular steady-state convective flow.

All molecules dissolved in the liquid are involved in circulation between temperature zones by being dragged by the convection flow. Traveling along temperature zones the molecules experience periodic temperature variations. For example, a double stranded DNA molecule being placed in the circular convection flow experience multiple cycles of heating and cooling. If the temperature of solution in the hot zone is sufficient to melt the DNA duplex and the temperature of the cold zone is favorable to maintain given nucleic acids in a double-stranded form then the circulation of nucleic acids in this convection flow results in repeatable denaturation and annealing cycles. Observation of the multiple cycles of ds-DNA denaturation and annealing can be performed by various methods, for example using fluorescent microscopy by registering the intensity of the non-specific DNA staining dyes placed in the reaction mixture along with the DNA sample.

A prototype heating instrument consists of two resistive Kapton foil heaters glued to the aluminum plates, and can be simultaneously used to provide differential heating for up to five fluidic chips. Two low-wattage power supplies power the heaters. The proposed amplification system is tolerant to heating element temperature inaccuracies in range of ±2° C., and does not require precise computer controlled hardware.

### 5. ENZYME-FREE LINEAR AMPLIFICATION EMBODIMENT

The present disclosure represents an enzyme-free amplification of target nucleic acid, in which amplicon concentration increases linearly with time (FIGS. 14A-B). Two surface regions are irreversibly functionalized with two DNA oligonucleotides, donor, D (comprising domain 1) and acceptor, A (comprising domains 4-5). The donor oligonucleotide functionalized to the surface region 1 is initially hybridized with a signal strand S comprising domains 2-3, which are complementary to domains 4-5, in such a way that the single-stranded domain 2 is exposed to solution and plays a role of a toehold sequence. The acceptor strand is irreversibly functionalized to surface region 2 and initially represents a single-stranded oligonucleotide. The surface regions 1 and 2 are localized in the temperature zone held at 35° C. (35° C. zone), at which the D-S duplex is designed to be highly stable over the time scale of a detection assay. Target molecule T (comprising domains 6-7) is introduced in the reaction solution and will be transferred by convection flow to the 35° C. zone of the reaction chamber. Target T binds to the signal S via domain 7 (the “toehold” domain) and displaces domain 3 from surface to the solution via toehold-mediated strand displacement mechanism. Then, Rayleigh-Bernard convection flow carries the duplex to a hot zone of the chamber (held at 85° C.), where the duplex dissociates. The two single-stranded molecules, the target strand T and the signal strand S are then transferred back into the chamber's 35° C. zone where strand S binds to the acceptor oligonucleotide A functionalized to the surface region 2. At the same time allowing the single-stranded target T catalytically displaces another signal S molecule from surface region 1, completing the catalytic cycle. This trigger should proceed continuously until the signal molecules S are completely transferred from surface region 1 to surface region 2.

The linear amplification scheme demonstrates the benefits of simultaneously using solid-phase sequestering of oligonucleotide reactants and the temperature-driven convection flow. Immobilization of the oligonucleotide reagents on the different surface regions allows avoiding false positive signal molecule release (spurious amplification) in the absence of the target sequence, while the thermal convection flow, beside spontaneous transport and improved mass transfer, induces target regeneration via melting of the target-signal complex (T-S). In contrast, changing the temperature of the entire solution is undesirable because it would lead to spontaneous dissociation of all oligonucleotides from the surface.

Labeling of the signal strand S with a fluorescent dye represent one approach for real-time monitoring of the linear amplification process. A decrease of the intensity of fluorescence registered form the of the surface region 1, as well as an increase of the intensity of fluorescence registered from the surface region 2 can effectively reflects how the reaction amplification reaction proceeds.

### 6. STOICHIOMETRIC DETECTION EMBODIMENT

To demonstrate that the enzyme-free amplification method exhibits multiple turnover, the inventors constructed a corresponding stoichiometric detection system using the convection device (FIGS. 15A-B). The stoichiometric system also includes two surface regions functionalized with a donor, Ds strand comprising domains 11-12 and an acceptor As strand comprising domain 16. The Ds strand is hybridized with a signal complex consisting of a bridge oligonucleotide Bs comprising domains 14-15 and a signal oligonucleotide Ss comprising domain 13. Domain 14 of the Bs strand and the As strand possess identical sequence. Target molecule T comprising domains 17-18 introduced in the reaction binds to the toehold domain 11 of the donor Ds and then displaces the signal complex into the solution. During this process target T binds the donor Ds and is unable to be regenerated in the chamber's hot zone in order to trigger the release of another signal complex from the surfaces region 1. The displaced signal complex is transferred by the convection flow into the 85° C. zone where it dissociates. After the dissociation the signal molecule Ss is transferred back to the 35° C. zone and is captured by the acceptor oligonucleotide As functionalized to the surface region 2. Thus, the amount of the captured signal strand Ss equals the amount of target T introduced into the system.

Real-time observation of the stoichiometric detection system can also be performed via simple labeling of the signal strand Ss with a fluorescent dye and registering the change surface region 2 fluorescence intensity. FIG. 16 shows the time-based decrease of fluorescence on surface region 1 for the linear amplification and stoichiometric detection systems, given identical initial quantities of detection target. The decrease of fluorescent signal is faster in the linear amplification system than in the linear amplification assay that supports the designed mechanism in which each target molecule is catalytically transferring multiple signal molecules from surface 1 to surface 2.

### 7. ENZYME-FREE EXPONENTIAL AMPLIFICATION EMBODIMENT

FIGS. 17A-B shows an embodiment of the present disclosure in which the detection target triggers the release of an amplicon product whose concentration exponentially increases with time, until reagents are consumed (FIGS. 17A-B). In this system, the two surface regions are functionalized with partially double-stranded oligonucleotide complexes: surface region 1 has a complex consisting of a single domain oligonucleotide 1 irreversibly attached to the surface and hybridized with an oligonucleotide S1 comprising domains 3-2 in such a way that the domain 3 remains single-stranded and acts as a toehold sequence. The surface region 2 mirrors surface region 1 and has a similar architecture of an oligonucleotide reagent functionalized to the surface: an oligonucleotide comprising domain 4 is irreversibly functionalized to the surface, and hybridized with an oligonucleotide S2 comprising domains 6-5 wherein domain 6 represents a single-stranded toehold. Injection of the target strand T, comprising domains 7 and 8 (identical in sequence to domains 6 and 5, respectively), into the reaction mixture results in the release of the oligonucleotide S1 from the surface region 1 in the form of double-strand amplicon.

Convection flow carries the duplex to the 85° C. zone where the duplex melts. Now two single-stranded oligonucleotides, the initial target T and released strand S1 flows back to the 35° C. zone where each of them triggers new release of oligonucleotide species from the surface. In particular, target oligonucleotide T catalytically releases second strand S1 from the surface region 1, while the initially released strand S1 triggers the release of the strand S2 from the second surface region. Thus, at the end of each convection flow cycle the amount of oligonucleotide species present in solution doubles, resulting in exponential accumulation of the amplicon species in solution phase.

### 8. ALTERNATIVE DETECTION METHODS

FIG. 14C presents one possible detection mechanism for assaying reaction progress. Alternative readout approaches also utilizing fluorescent microscopy are possible. Non-sequence-specific intercalating nucleic acid staining dyes, such as SybrGreen and Syto dyes, can be used to indicate the total amount of accumulated double-stranded product (FIG. 18). Because the acceptor strand A (domains 4-5) immobilized on the surface region 2 is in single-stranded form at the beginning of the reaction, the intercalating dye would have a low affinity to the strand A. In the course of the reaction more and more strands S (domains 2-3) will hybridize with the surface functionalized strand A (domains 4-5). The newly formed complexes A-S now would be efficiently stained with the dye that would cause an increase of the surface region 2 fluorescence. The opposite situation can be potentially observed on the surface region 1.

Application of the FRET-based detection technique is illustrated on the example of exponential amplification (FIG. 19). For example, irreversibly labeled with a fluorescent dye surface immobilized strands (domain 1 is shown labeled) can be efficiently quenched with a quencher functionalized signal strands (the strand with domains 2-3 is shown with a fluorescent quencher) before the target is added. An addition of the target will result in exponential release of the signal strands forms the surface and lighting up of the surface immobilized strands.

### 9. REAL-TIME DETECTION OF CONVECTION FLOW PCR

Another application the composition claimed in the present disclosure is that the composition can be used as an efficient mean for surface-based real-time monitoring of an enzymatic nucleic acid amplification process proceeding in the solution. There are no reported examples of real-time monitoring of convection-based PCR using surface functionalized probes.

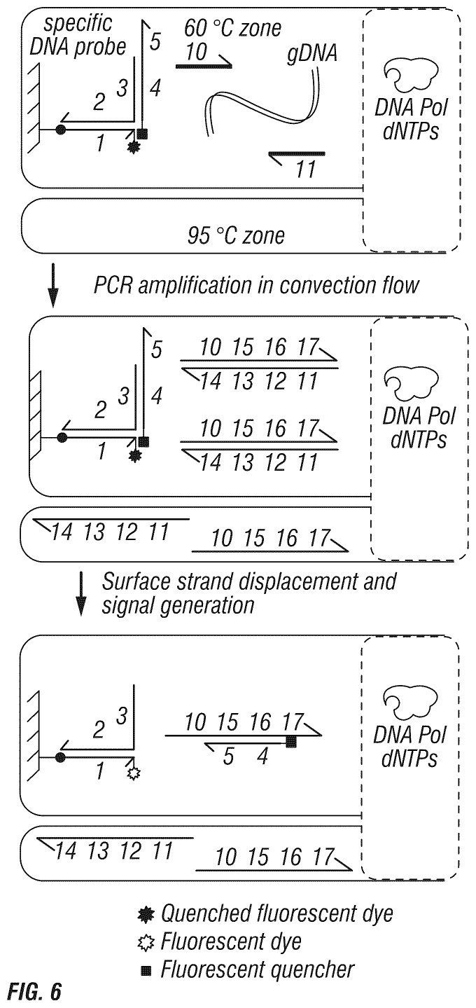

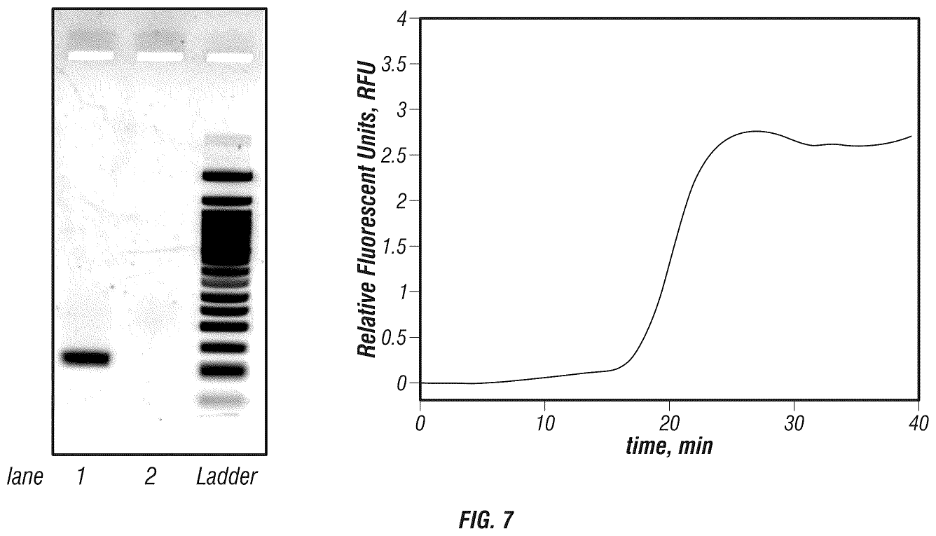

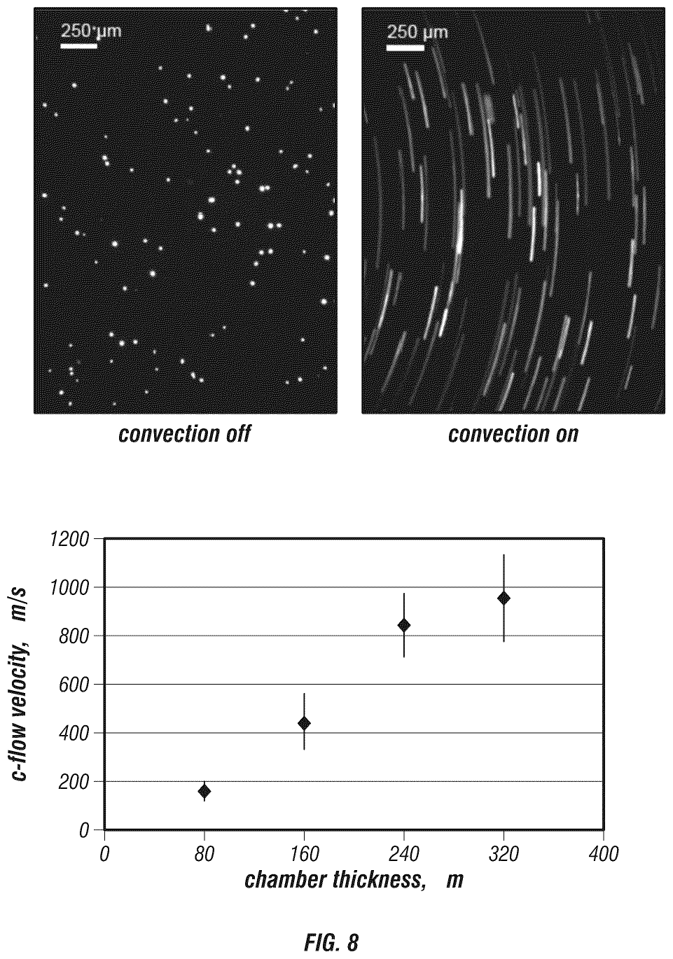

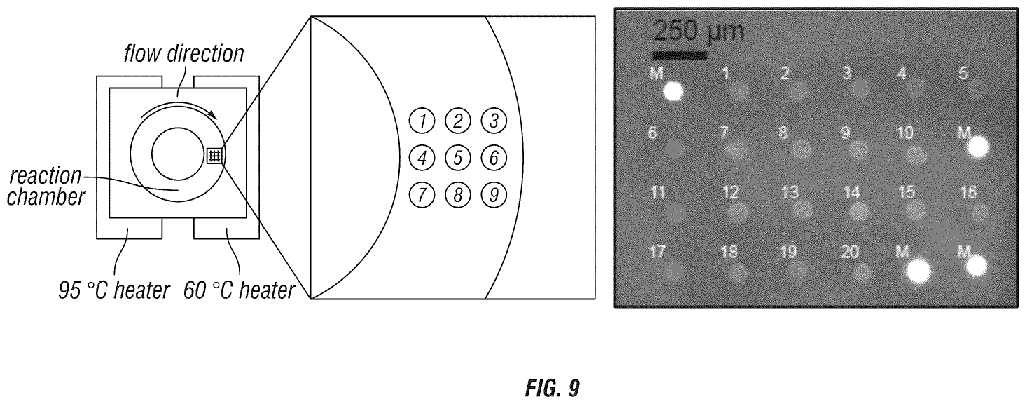

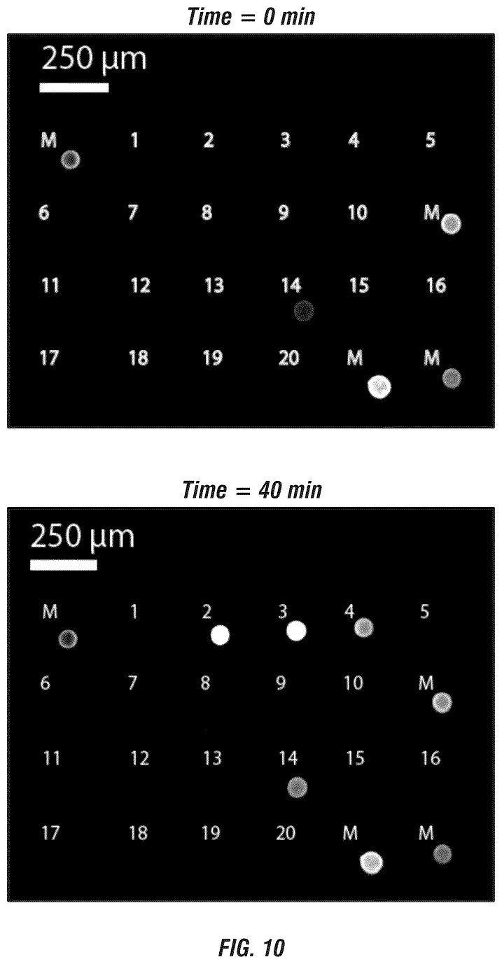

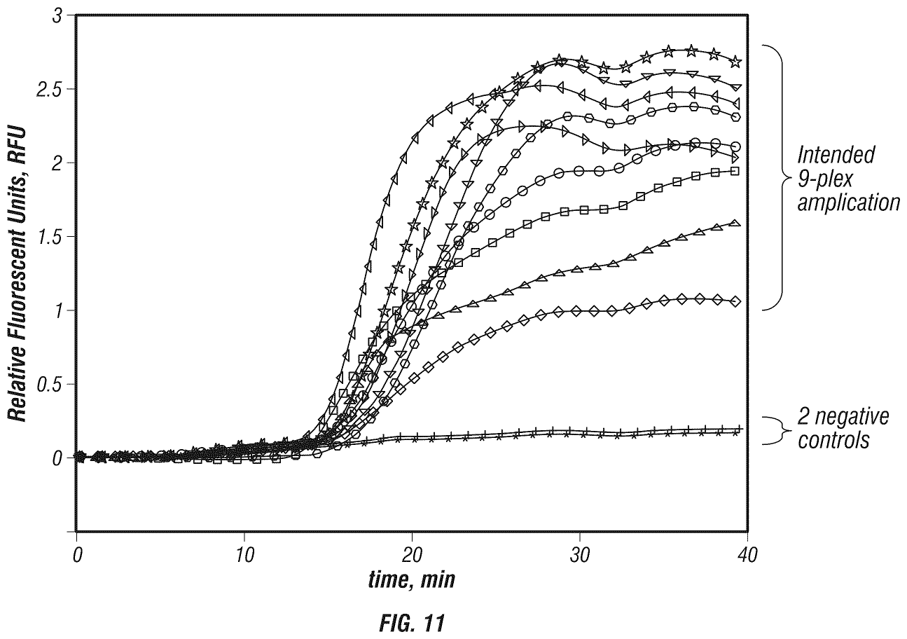

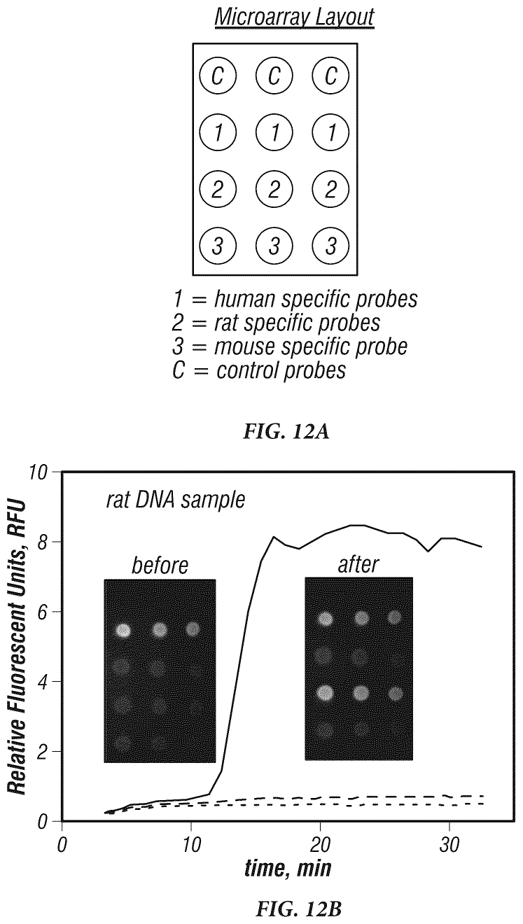

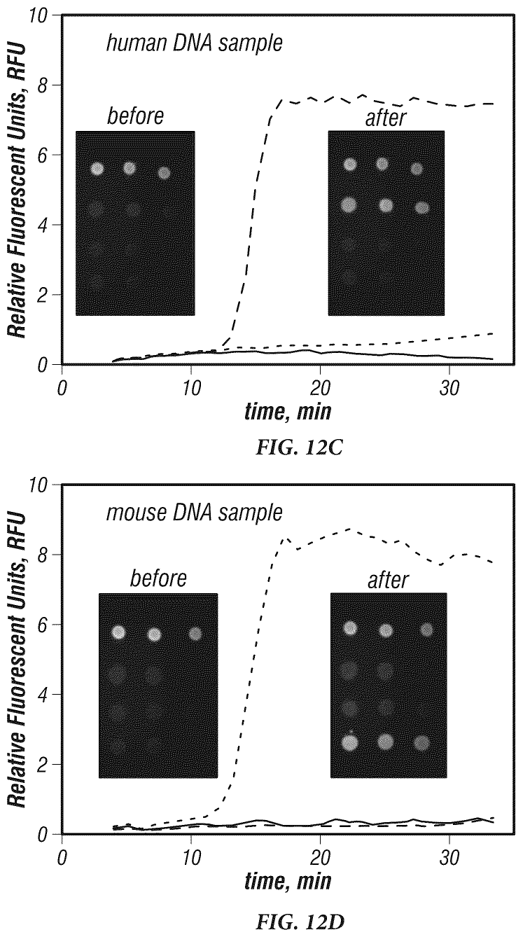

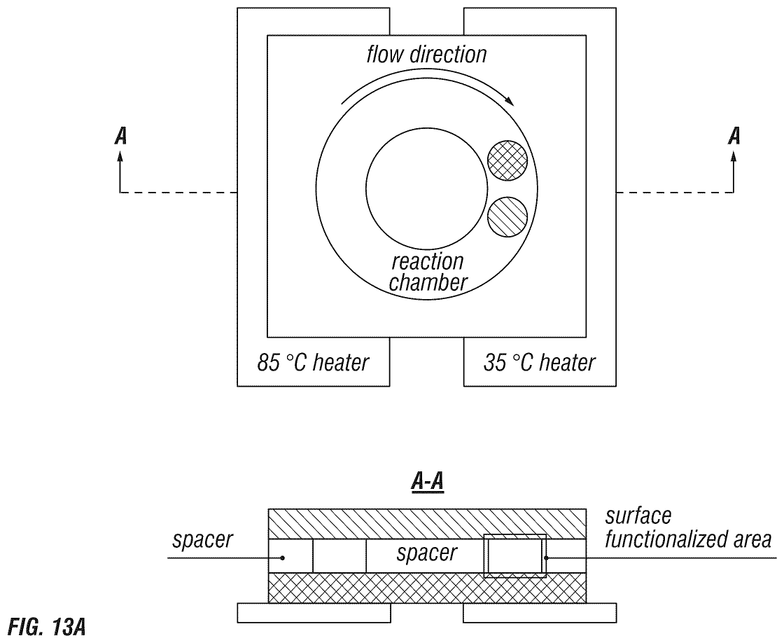

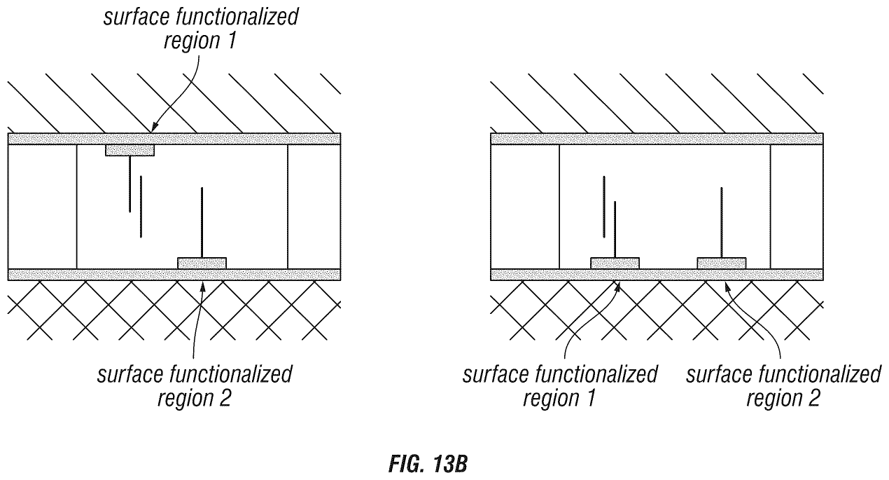

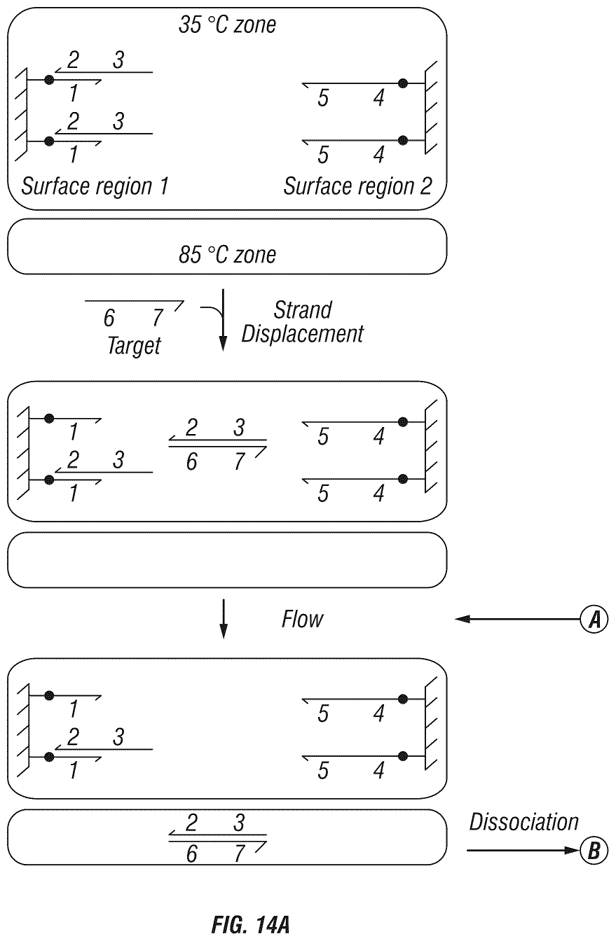

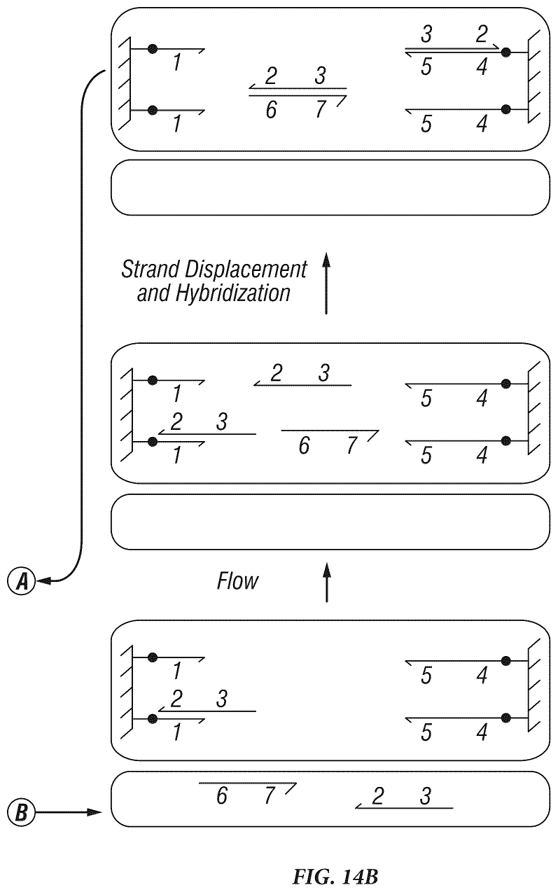

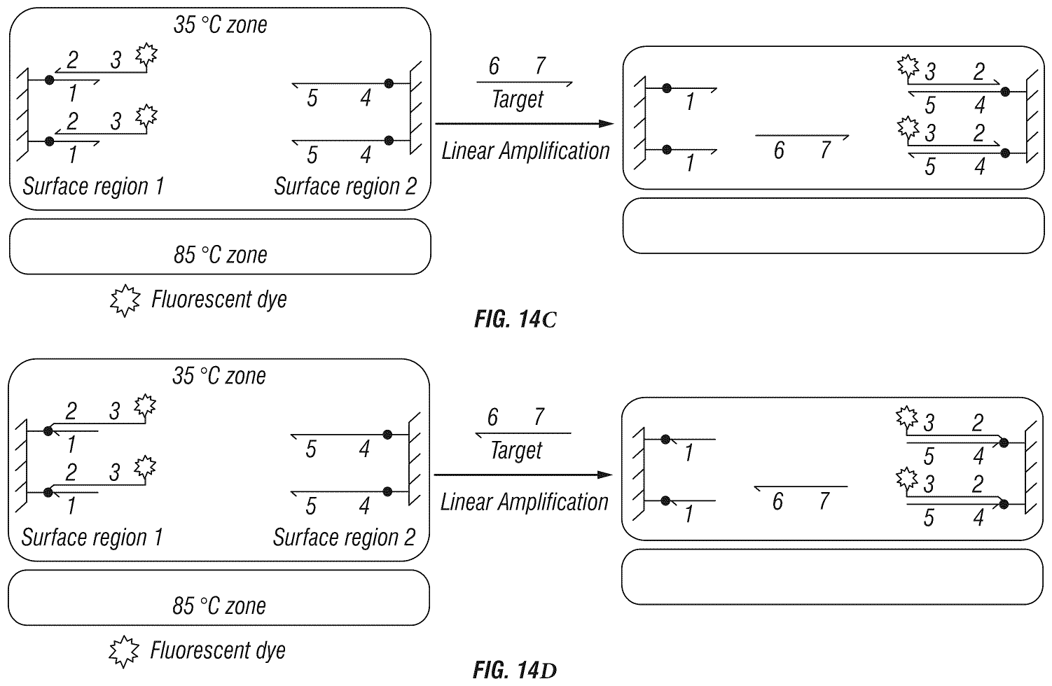

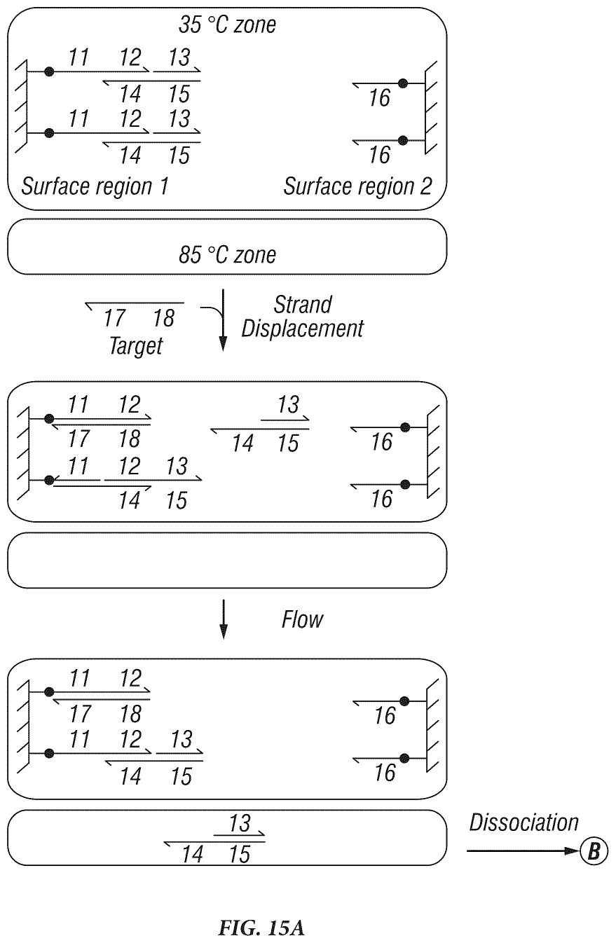

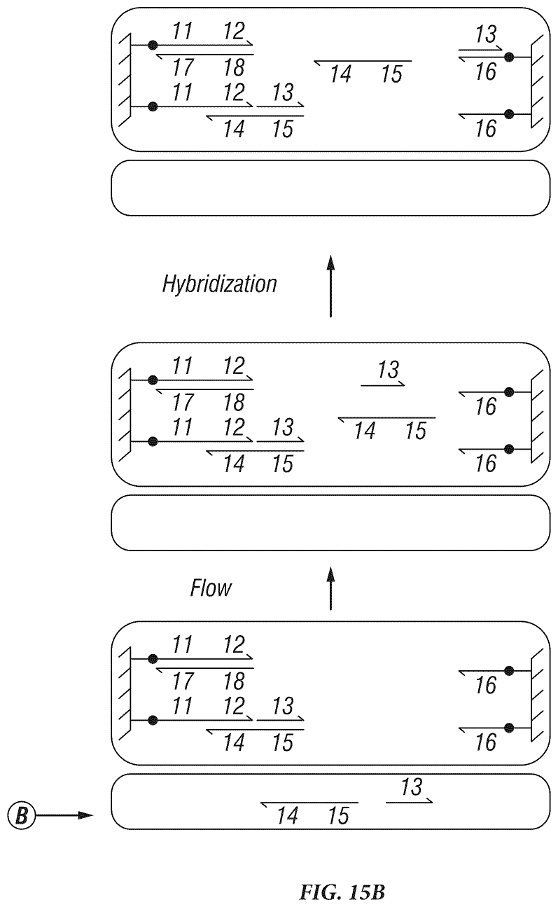

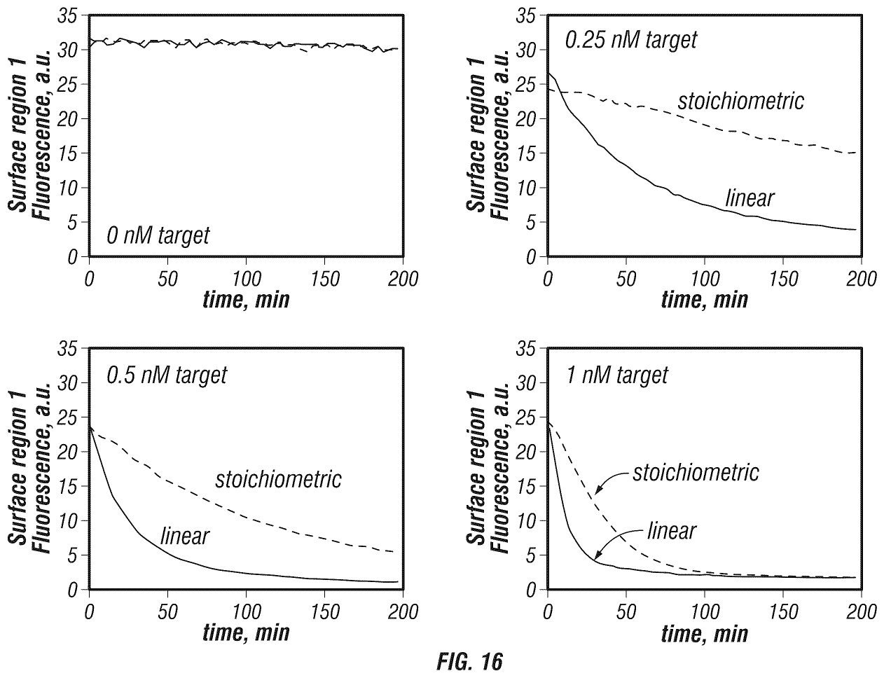

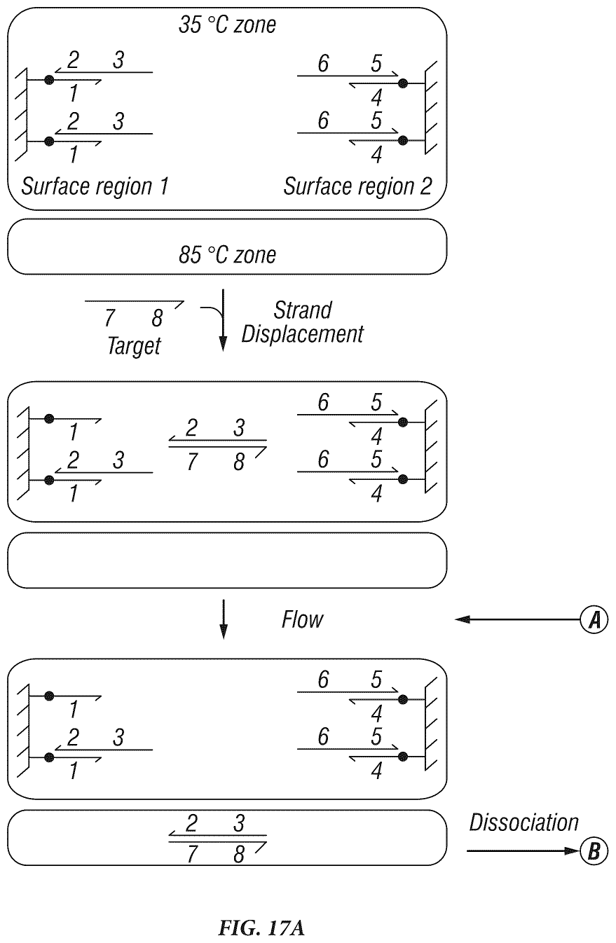

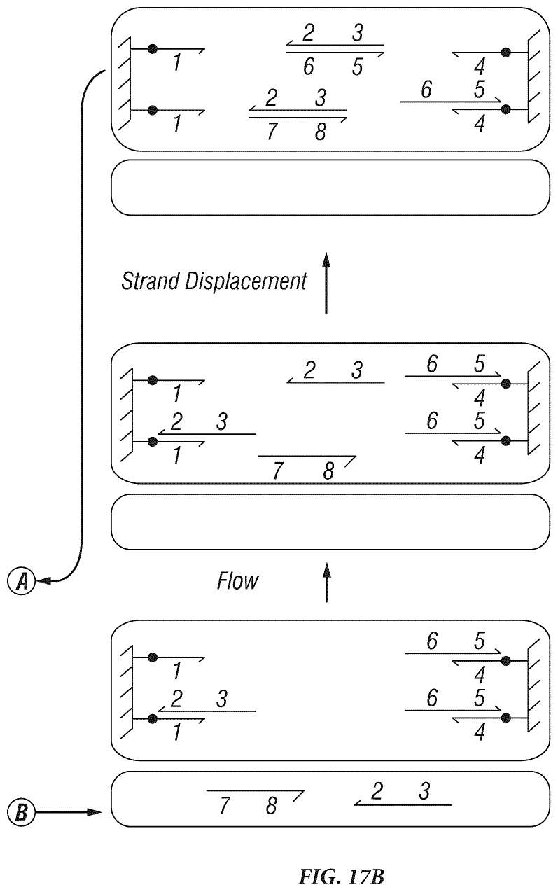

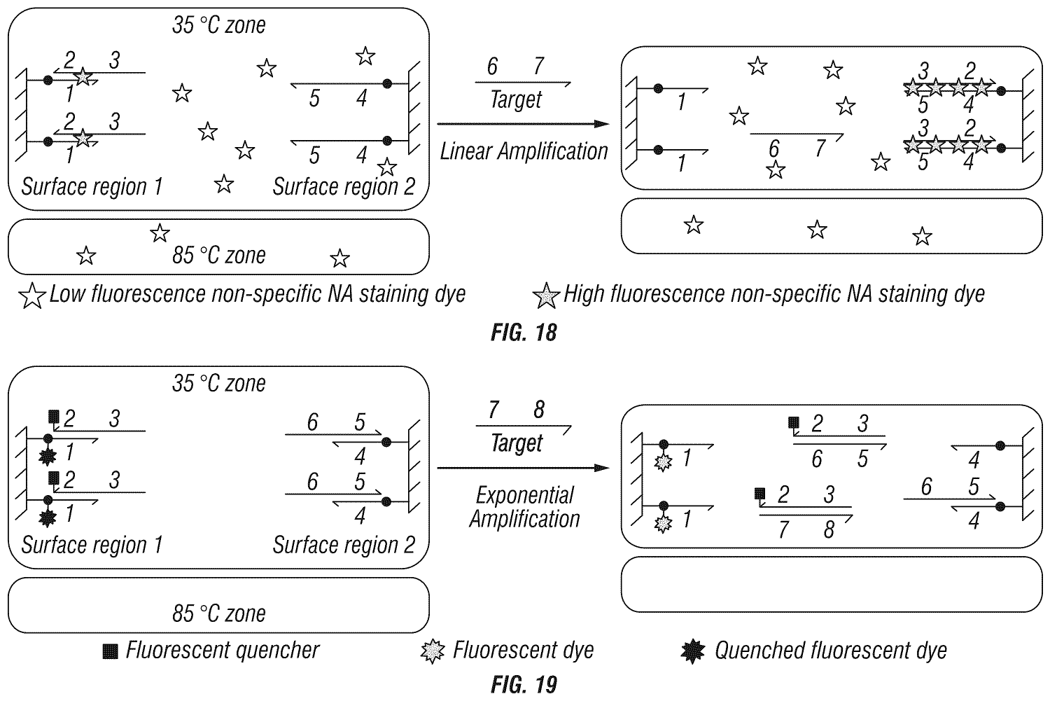

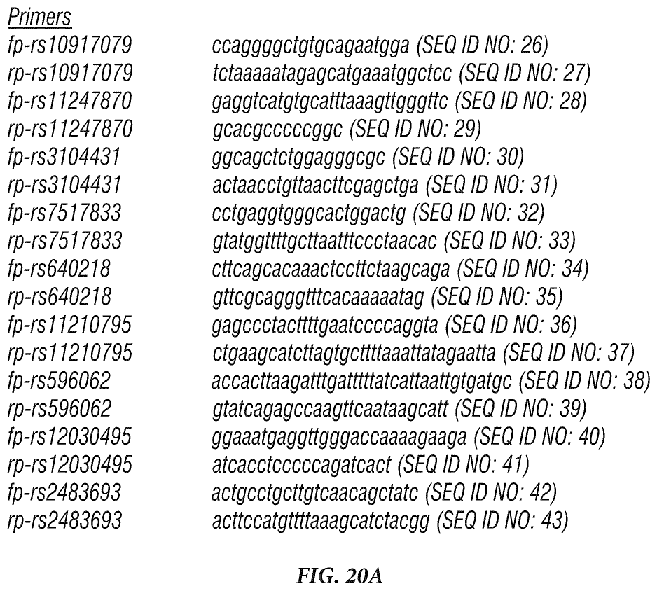

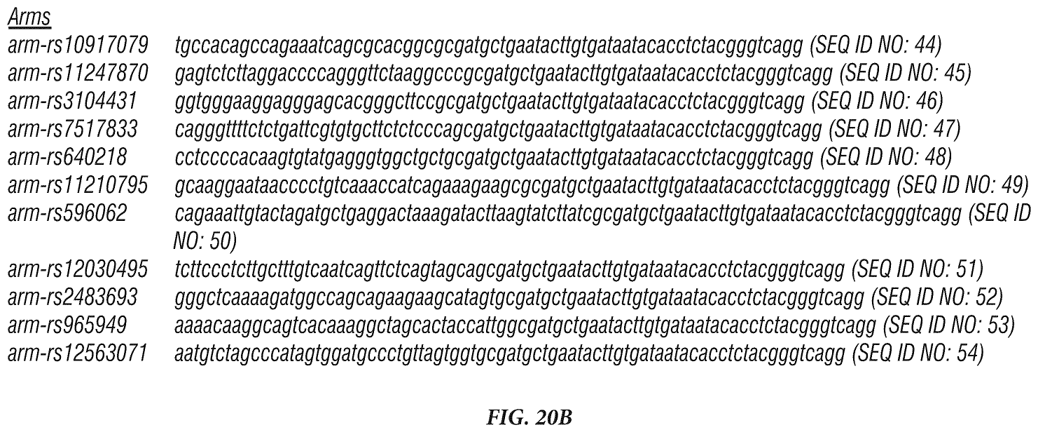

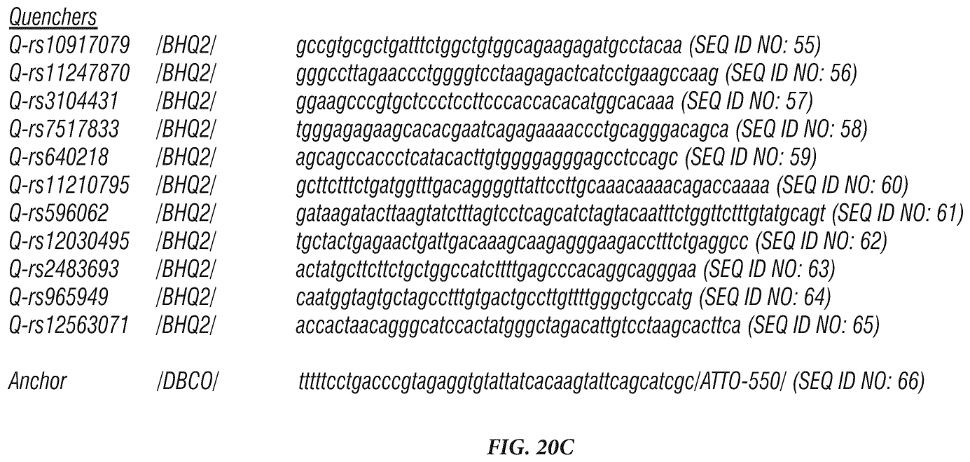

FIG. 6 shows an approach utilizing the claimed composition for real-time monitoring of the PCR reaction performed in the temperature-driven convection flow. Initially a surface region 1 residing in 60° C. zone is functionalized with an Anchor oligonucleotide comprising domain 1 and a fluorescent dye. An Arm oligonucleotide comprising domains 3-2 is hybridized to the Anchor oligonucleotide through its 2 domain. The Anchor-Arm oligonucleotide complex is in turn hybridized with a Quencher oligonucleotide comprising domains 4 and 5 and a fluorescent quencher through its domain 4; domain 5 is single-stranded. The reaction solution comprises reagents for enzymatic extension of oligonucleotide primers (domains 10 and 11), such as a DNA polymerase, mixture of deoxynucleotide triphosphates, divalent ions (Mg²⁺). After injection of a target molecule 5 (gDNA), it spontaneously melts in the 95° C. Then the melted gDNA is transferred into the 60° C. zone by the convection flow where primers anneal to their specific target sequences and extend by DNA polymerase that leads to formation of the double stranded amplicon molecules comprising domains 10-15-16-17 in forward strand and 11-12-13-14 in reverse strand. The amplicon molecules melt in the 95° C. and flow back to the 60° C. zone where forward strand 10-15-16-17 displaces the Quencher oligonucleotide form the surface functionalized complex Anchor-Arm resulting in increasing of the fluorescent signal registered form the surface region 1.

### 10. SIMULTANEOUS MONITORING OF MULTIPLE TARGET SEQUENCES

The proposed compositions and methods in this disclosure allow for simultaneous monitoring of the amplification (either enzyme-free or enzyme-based) of multiple nucleic acid target sequences. Spatial patterning of different oligonucleotide probes at different surface regions allows an array- or camera-based readout to provide independent information on the amplicon concentrations of each target amplification reaction.

### 11. EXEMPLARY OLIGONUCLEOTIDES

The following oligonucleotide sequences are provided by way of example, but not limitation:

### Linear Amplification System Oligonucleotides

1
(SEQ ID NO: 17)
/5Hexynyl/TTTTTCCGTAGAGGTGTATTATCACAAGTATT
2-3
(SEQ ID NO: 18)
/56-TAMN/TGGATGCTG-AATACTTGTGATAATACACCTCTACGG
4-5
(SEQ ID NO: 19)
/5Hexynyl/TTTTTCCGTAGAGGTGTATTATCACAAGTATT-
CAGCATCCA
6-7
(SEQ ID NO: 20)
CCGTAGAGGTGTATTATCACAAGTATT-CAGCATCCA

11-12
(SEQ ID NO: 21)
/5Hexynyl/TTTTTTGTCAACC-ATCATCGTTCGTACCACAGTGTTC
AG
13
(SEQ ID NO: 22)
/56-TAMN/TGGATGCTGAATACTTGTGATAATACACCTCTACGG
14-15
(SEQ ID NO: 23)
CCGTAGAGGTGTATTATCACAAGTATTCAGCATCCA-
CTGAACACTGTGGTACGAACGATGA
16
(SEQ ID NO: 24)
/5Hexynyl/TTTTTCCGTAGAGGTGTATTATCACAAGTATTCAGCAT
CCA
17-18
(SEQ ID NO: 25)
CTGAACACTGTGGTACGAACGATGAT-GGTTGACA

### 12. REFERENCES

The following references, to the extent that they provide exemplary procedural or other details supplementary to those set forth herein, are specifically incorporated herein by reference.
1. Eckert K, Bestehorn M, Thess A. Square cells in surface-tension-driven Benard convection: experiment and theory. J Fluid Mech. 1998; 356:155-197.
2. Krishnan M, Ugaz V M, Burns M a. PCR in a Rayleigh-Bénard convection cell. Science. 2002; 298(5594):793. doi:10.1126/science.298.5594.793.
3. Hennig M, Braun D. Convective polymerase chain reaction around micro-immersion heater. Appl Phys Lett. 2005; 87(18):1-3. doi:10.1063/1.2051787.
4. Hsieh Y-F, Yonezawa E, Kuo L-S, Yeh S-H, Chen P-J, Chen P-H. Polymerase chain reaction with phase change as intrinsic thermal control. Appl Phys Lett. 2013; 102(May 2015):2013-2016. doi:10.1063/1.4803442.
5. Priye A, Hassan Y A, Ugaz V M. Microscale Chaotic Advection Enables Robust Convective DNA Replication. Anal Chem. 2013; 85(21):10536-10541. doi:10.1021/ac402611s.
6. Agrawal N, Hassan Y a., Ugaz V M. A pocket-sized convective PCR thermocycler. Angew Chemie-Int Ed. 2007; 46(23):4316-4319. doi:10.1002/anie.200700306.
7. Hsieh Y F, Lee D S, Chen P H, et al. A real-time convective PCR machine in a capillary tube instrumented with a CCD-based fluorometer. Sensors Actuators, B Chem. 2013; 183:434-440. doi:10.1016/j.snb.2013.04.003.
8. Chen J, Lozach J, Garcia E W, et al. Highly sensitive and specific microRNA expression profiling using BeadArray technology. Nucleic Acids Res. 2008; 36(14):e87. doi:10.1093/nar/gkn387.
9. Anzai Y, Saito S, Fujimoto K, Kinoshita K, Kato F. Detection and Identification of Species with Bacterial Cells Using a Plastic DNA Array. J Heal Sci. 2008; 54(2):229-234. doi:10.1248/jhs.54.229.
10. Andresen H, Grötzinger C, Zarse K, Kreuzer O J, Ehrentreich-Förster E, Bier F F. Functional peptide microarrays for specific and sensitive antibody diagnostics. Proteomics. 2006; 6(5):1376-1384. doi:10.1002/pmic.200500343.
11. Chung M Y, Kim Y-W, Bae S M, et al. Development of a bead-based multiplex genotyping method for diagnostic characterization of HPV infection. PLoS One. 2012; 7(2):e32259. doi:10.1371/journal.pone.0032259.
12. Dai X, Yang W, Firlar E, Marras S a. E, Libera M. Surface-patterned microgel-tethered molecular beacons. Soft Matter. 2012; 8(11):3067. doi:10.1039/clsm06702h.
13. Hoheisel J D. Microarray technology: beyond transcript profiling and genotype analysis. Nat Rev Genet. 2006; 7(3):200-210. doi:10.1038/nrg1809.
14. Gitan R S, Shi H, Chen C M, Yan P S, Huang T H M. Methylation-specific oligonucleotide microarray: A new potential for high-throughput methylation analysis. Genome Res. 2002; 12(1):158-164. doi:10.1101/gr.202801.
15. Iwasaki H. Accuracy of Genotyping for Single Nucleotide Polymorphisms by a Microarray-Based Single Nucleotide Polymorphism Typing Method Involving Hybridization of Short Allele-Specific Oligonucleotides. DNA Res. 2002; 9(2):59-62. doi:10.1093/dnares/9.2.59.
16. Jenison R, Jaeckel H, Klonoski J, Latorra D, Wiens J. Rapid amplification/detection of nucleic acid targets utilizing a HDA/thin film biosensor. Analyst. 2014; 3763-3769. doi:10.1039/c4an00418c.
17. Chudinov A V., Kolganova N a., Egorov A E, et al. Bridge DNA amplification of cancer-associated genes on cross-linked agarose microbeads. Microchim Acta. 2014; 182(3-4):557-563. doi:10.1007/s00604-014-1357-8.
18. Quan J, Saaem I, Tang N, et al. Parallel on-chip gene synthesis and application to optimization of protein expression. Nat Biotechnol. 2011; 29(5):449-452. doi:10.1038/nbt.1847.
19. Yurke B, Turberfield A J, Mills A P, Simmel F C, Neumann J L. A DNA-fuelled molecular machine made of DNA. Nature. 2000; 406(6796):605-608. doi:10.1038/35020524.
20. Zhang D Y. Cooperative hybridization of oligonucleotides. J Am Chem Soc. 2011; 133(4):1077-1086. doi:10.1021/ja109089q.
21. Zhang D Y, Chen S X, Yin P. Optimizing the specificity of nucleic acid hybridization. Nat Chem. 2012; 4(3):208-214. doi:10.1038/nchem.1246.
22. Zhang Z, Zeng D, Ma H, et al. A DNA-Origami chip platform for label-free SNP genotyping using toehold-mediated strand displacement. Small. 2010; 6(17):1854-1858. doi:10.1002/smll.201000908.
23. Zhao C, Song Y, Ren J, Qu X. A DNA nanomachine induced by single-walled carbon nanotubes on gold surface. Biomaterials. 2009; 30(9):1739-1745. doi:10.1016/j.biomaterials.2008.12.034.
24. Zhang X-B, Kong R-M, Lu Y. Metal ion sensors based on DNAzymes and related DNA molecules. Annu Rev Anal Chem (Palo Alto Calif). 2011; 4:105-128. doi:10.1146/annurev.anchem.111808.073617.
25. Yan H, Zhang X, Shen Z, Seeman N C. A robust DNA mechanical device controlled by hybridization topology. Nature. 2002; 415(6867):62-65. doi:10.1038/415062a.
26. Turberfield A, Mitchell J, Yurke B, Mills A, Blakey M, Simmel F. DNA Fuel for Free-Running Nanomachines. Phys Rev Lett. 2003; 90(11):1-4. doi:10.1103/PhysRevLett.90.118102.
27. Shlyahovsky B, Li Y, Lioubashevski O, Elbaz J, Willner I. Logic Gates and Antisense DNA Devices Scaffold. ACS Nano. 2009; 3(7):1831-1843.
U.S. Pat. No. 6,586,233 B2. Convectively driven PCR thermal-cycling, William J. Benett, James B. Richards, Fred P. Milanovich
U.S. Pat. No. 8,735,103 B2. Natural convection-driven PCR apparatus and method using disposable polymer chip. Kwang-Hyo Chung, Dae-Sik Lee, Hyeon-Bong Pyo, Seon-Hee Park
U.S. Pat. No. 8,187,813 B2. Methods and apparatuses for convective polymerase chain reaction (PCR). Pei-Jer Chen, Ping-Hei Chen, Wen-Pin Chou, Yi-Fan Hsieh, Shiou-Hwei Yeh
U.S. Pat. No. 8,043,810 B2. Analyte detection using autocatalytic chain reactions. John H. Reif, Peng Yin, Thomas H. LaBean, Geetha Shetty, Erik A. Schultes
5. U.S. Pat. No. 8,110,353 B2. Engineered toehold reactions and networks. David Zhang, Andrew J. Turberfield, Erik Winfree
6. U.S. Pat. No. 8,630,809 B2. System and method for propagating information using modified nucleic acids. Daniel J. Kleinbaum
7. U.S. Pat. No. 6,300,070 B1. Solid phase methods for amplifying multiple nucleic acids. T. Christian Boles, Ezra S. Abrams
8. U.S. Pat. No. 7,399,584 B2. Method of comparing a target nucleic acid and a reference nucleic acid. Maureen T. Cronin, Charles Garrett Miyada, Earl A. Hubbell, Mark Chee, Stephen P. A. Fodor, Xiaohua C. Huang, Robert J. Lipshutz, Peter E. Lobban, MacDonald S. Morris, Edward L. Sheldon
9. U.S. Pat. No. 6,642,000 B2. PCR amplification on microarrays of gel immobilized oligonucleotides. Boris Strizhkov, Sergei Tillib, Vladimir Mikhailovich, Andrei Mirzabekov.
10. U.S. Pat. No. 7,846,656 B2. Composition for polymerizing immobilization of biological molecules and method for producing said composition. Andrei Mirzabekov, Alla Rubina, Sergei Pankov.

1-35. (canceled)

36. A method of amplifying a target nucleic acid comprising
(a) providing a fluidic reaction chamber comprising:
a first surface,
a second surface that does not contact the first surface, wherein the first and second surfaces face each other,
a material contacting the first surface and the second surface and that forms an outer boundary of the reaction chamber, and
a material contacting the first surface and the second surface and that forms an inner boundary of the reaction chamber,
wherein the first surface comprises a plurality of oligonucleotide complexes, wherein the oligonucleotide complexes each comprise:
a first oligonucleotide comprising a first DNA sequence and a linking moiety for irreversibly linking the first oligonucleotide to the surface, and
a second oligonucleotide comprising a second DNA sequence and a third DNA sequence, wherein the second DNA sequence is complementary to the first DNA sequence and is hybridized thereto, wherein said second DNA oligonucleotide is not irreversibly linked to the first surface, and optionally does not comprise a fluorescent moiety,
wherein the fluidic reaction chamber is in operable relationship to a first and a second heat source, wherein the first and second heat sources are capable of applying differing first and second heat levels to the fluidic reaction chamber, wherein the first and second heat levels are not the same;
(b) introducing into the fluidic reaction chamber a fluid comprising a target nucleic acid sequence, a DNA polymerase, dNTPs, and a polymerase chain reaction (PCR) buffer; and
(c) applying first and second heat levels to the fluidic reaction chamber.

37. The method of claim 36, further comprising detecting amplification of the target nucleic acid.

38. (canceled)

39. The method of claim 36 wherein each of the second DNA sequences are not identical.

40. (canceled)

41. The method of claim 37, wherein each of the plurality of the oligonucleotide complexes are located in spatially discrete regions on the surface.

42. The method of claim 36, wherein each of the first oligonucleotides comprise a fluorescent moiety.

43. The method of claim 42, wherein each of the second oligonucleotides comprise a fluorescence quencher moiety.

44. The method of claim 42, wherein each of the spatially discrete regions further comprises a third oligonucleotide comprising a fourth DNA sequence and a fifth DNA sequence, wherein the fourth DNA sequence is complementary to the third DNA sequence, wherein each of the third oligonucleotides comprises a fluorescence quencher moiety.

45-46. (canceled)

47. The method of claim 36, wherein each of the oligonucleotides has a length of between about 5 and about 120 nucleotides.

48-50. (canceled)

51. The method of claim 36, wherein said fluid further comprises a non-specific nucleic acid staining dye.

52. The method of claim 36, wherein the fluidic reaction chamber is circular, oval, square, triangular, rectangular, hexagonal, octagonal, rhomboid, or trapezoid.

53. The method of claim 36, wherein the fluidic reaction chamber is not at a uniform temperature, and wherein the warmest region of the reaction chamber is between about 80° C. and about 100° C., and the coldest region of the reaction chamber is between about 45° C. and about 75° C.

54-90. (canceled)

91. A method of amplifying a target nucleic acid comprising:
(a) providing a fluidic reaction chamber comprising:
a first surface,
a second surface that does not contact the first surface, wherein the first and second surfaces face each other,
a material contacting the first surface and the second surface and that forms an outer boundary of the reaction chamber, and
a material contacting the first surface and the second surface and that forms and inner boundary of the reaction chamber, wherein
(i) the first surface comprises a plurality of oligonucleotide complexes, wherein the oligonucleotide complexes are located in spatially discrete locations of the first surface region and each comprise:
a first oligonucleotide comprising a first DNA sequence and a linking moiety for irreversibly linking the first oligonucleotide to the first surface region, and
a second oligonucleotide comprising a second DNA sequence and a third DNA sequence, wherein the second DNA sequence is complementary to the first DNA sequence, and
the second surface region comprises a plurality of oligonucleotide complexes, wherein the oligonucleotide complexes are located in spatially discrete locations of the second surface region and each comprise:
a third oligonucleotide comprising a fourth DNA sequence and a linking moiety for irreversibly linking the third oligonucleotide to the second surface region, and
wherein the fourth DNA sequence is complementary to the second DNA sequence, the third DNA sequence, or a combination of at least six continuous nucleotides of the second DNA sequence and six continuous nucleotides of the third DNA sequence; or
(ii) the first surface region comprises a plurality of oligonucleotide complexes, wherein the oligonucleotide complexes are located in spatially discrete locations of the first surface region and each comprise:
a first oligonucleotide comprising a first DNA sequence and a linking moiety for irreversibly linking the first oligonucleotide to the first surface region, and
a second oligonucleotide comprising a second DNA sequence and a third DNA sequence, wherein the second DNA sequence is complementary to the first DNA sequence, and
the second surface region comprises a plurality of oligonucleotide complexes, wherein the oligonucleotide complexes are located in spatially discrete locations of the second surface region and each comprise:
a third oligonucleotide comprising a fourth DNA sequence and a linking moiety for irreversibly linking the third oligonucleotide to the second surface region, and
a fourth oligonucleotide comprising a fifth DNA sequence and a sixth DNA sequence, wherein the fifth DNA sequence is complementary to the fourth DNA sequence, and
wherein the second DNA sequence is complementary to the fifth DNA sequence or is complementary to the sixth DNA sequence,
wherein the fluidic reaction chamber is in operable relationship to a first and a second heat source, wherein the first and second heat source are capable of applying differing first and second heat levels to the fluidic reaction chamber, wherein the first and second heat levels are not the same;
(b) introducing into the fluidic reaction chamber a fluid comprising a target nucleic acid sequence; and
(c) applying first and second heat levels to the fluidic reaction chamber.

92. The method of claim 91, further comprising detecting amplification of the target nucleic acid.

93. The method of claim 91, wherein the first or second oligonucleotide comprises a fluorescent moiety.

94. The method of claim 91, wherein the first or second oligonucleotide comprises a fluorescence quencher.

95. (canceled)

96. The method of claim 91, wherein each of the second DNA sequences are not identical.

97. The method of claim 91, wherein each of the plurality of the oligonucleotide complexes are located in spatially discrete regions on the surface.

98-99. (canceled)

100. The method of claim 91, wherein each of the oligonucleotides has a length of between about 5 and about 120 nucleotides.

101-105. (canceled)

106. The method of claim 91, wherein the fluidic reaction chamber is circular, oval, square, rectangular, triangular, hexagonal, octagonal, rhomboid, or trapezoid.

107. The method of claim 91, wherein the fluidic reaction chamber is not at a uniform temperature, and wherein the warmest region of the reaction chamber is between about 80° C. and about 100° C., and the coldest region of the reaction chamber is between about 45° C. and about 75° C.

108-110. (canceled)


SEQUENCE LISTING (66 sequences)

SEQ ID NO: 1 - DNA, 31 bp, synthetic construct
  misc_feature 1..31: note = Synthetic oligonucleotide
  source 1..31: mol_type = other DNA
tttttggatgctgaatacttgtgataataca

SEQ ID NO: 2 - DNA, 36 bp, synthetic construct
  misc_feature 1..36: note = Synthetic oligonucleotide
  source 1..36: mol_type = other DNA
ccgtagaggtgtattatcacaagtattcagcatcca

SEQ ID NO: 3 - DNA, 36 bp, synthetic construct
  misc_feature 1..36: note = Synthetic oligonucleotide
  source 1..36: mol_type = other DNA
tggatgctgaatacttgtgataatacacctctacgg

SEQ ID NO: 4 - DNA, 28 bp, synthetic construct
  misc_feature 1..28: note = Synthetic primer
  source 1..28: mol_type = other DNA
gttaacagattggaggtagtagcatttt

SEQ ID NO: 5 - DNA, 20 bp, synthetic construct
  misc_feature 1..20: note = Synthetic primer
  source 1..20: mol_type = other DNA
tctatcaccaccccccaact

SEQ ID NO: 6 - DNA, 27 bp, synthetic construct
  misc_feature 1..27: note = Synthetic primer
  source 1..27: mol_type = other DNA
caggtattttggggtatgattatggtt

SEQ ID NO: 7 - DNA, 25 bp, synthetic construct
  misc_feature 1..25: note = Synthetic primer
  source 1..25: mol_type = other DNA
ccaacagaatttaccaggaaacaca

SEQ ID NO: 8 - DNA, 25 bp, synthetic construct
  misc_feature 1..25: note = Synthetic primer
  source 1..25: mol_type = other DNA
caatacggccaaatctgaaagacaa

SEQ ID NO: 9 - DNA, 21 bp, synthetic construct
  misc_feature 1..21: note = Synthetic primer
  source 1..21: mol_type = other DNA
ctgcaggttctccacacctat

SEQ ID NO: 10 - DNA, 77 bp, synthetic construct
  misc_feature 1..77: note = Synthetic oligonucleotide
  source 1..77: mol_type = other DNA
agcagtgcttgctgttccttagaattttgccttgtgcgatgctgaatacttgtgataatacacctctacgggtcagg

SEQ ID NO: 11 - DNA, 72 bp, synthetic construct
  misc_feature 1..72: note = Synthetic oligonucleotide
  source 1..72: mol_type = other DNA
ctggttcttactgcagggcgtgggaggagcgcgatgctgaatacttgtgataatacacctctacgggtcagg

SEQ ID NO: 12 - DNA, 74 bp, synthetic construct
  misc_feature 1..74: note = Synthetic oligonucleotide
  source 1..74: mol_type = other DNA
gatagcctggggctcactacagacccatgagggcgatgctgaatacttgtgataatacacctctacgggtcagg

SEQ ID NO: 13 - DNA, 49 bp, synthetic construct
  misc_feature 1..49: note = Synthetic oligonucleotide
  source 1..49: mol_type = other DNA
acaaggcaaaattctaaggaacagcaagcactgctgcacgatcaggggt

SEQ ID NO: 14 - DNA, 46 bp, synthetic construct
  misc_feature 1..46: note = Synthetic oligonucleotide
  source 1..46: mol_type = other DNA
gctcctcccacgccctgcagtaagaaccagaccccagcctttacac

SEQ ID NO: 15 - DNA, 51 bp, synthetic construct
  misc_feature 1..51: note = Synthetic oligonucleotide
  source 1..51: mol_type = other DNA
cctcatgggtctgtagtgagccccaggctatctcatgttcttcagagtgga

SEQ ID NO: 16 - DNA, 47 bp, synthetic construct
  misc_feature 1..47: note = Synthetic oligonucleotide
  source 1..47: mol_type = other DNA
tttttcctgacccgtagaggtgtattatcacaagtattcagcatcgc

SEQ ID NO: 17 - DNA, 32 bp, synthetic construct
  misc_feature 1..32: note = Synthetic oligonucleotide
  source 1..32: mol_type = other DNA
tttttccgtagaggtgtattatcacaagtatt

SEQ ID NO: 18 - DNA, 36 bp, synthetic construct; identical to SEQ ID NO: 3
  misc_feature 1..36: note = Synthetic oligonucleotide
  source 1..36: mol_type = other DNA

SEQ ID NO: 19 - DNA, 41 bp, synthetic construct
  misc_feature 1..41: note = Synthetic oligonucleotide
  source 1..41: mol_type = other DNA
tttttccgtagaggtgtattatcacaagtattcagcatcca

SEQ ID NO: 20 - DNA, 36 bp, synthetic construct; identical to SEQ ID NO: 2
  misc_feature 1..36: note = Synthetic oligonucleotide
  source 1..36: mol_type = other DNA

SEQ ID NO: 21 - DNA, 39 bp, synthetic construct
  misc_feature 1..39: note = Synthetic oligonucleotide
  source 1..39: mol_type = other DNA
ttttttgtcaaccatcatcgttcgtaccacagtgttcag

SEQ ID NO: 22 - DNA, 36 bp, synthetic construct; identical to SEQ ID NO: 3
  misc_feature 1..36: note = Synthetic oligonucleotide
  source 1..36: mol_type = other DNA

SEQ ID NO: 23 - DNA, 61 bp, synthetic construct
  misc_feature 1..61: note = Synthetic oligonucleotide
  source 1..61: mol_type = other DNA
ccgtagaggtgtattatcacaagtattcagcatccactgaacactgtggtacgaacgatga

SEQ ID NO: 24 - DNA, 41 bp, synthetic construct; identical to SEQ ID NO: 19
  misc_feature 1..41: note = Synthetic oligonucleotide
  source 1..41: mol_type = other DNA

SEQ ID NO: 25 - DNA, 34 bp, synthetic construct
  misc_feature 1..34: note = Synthetic oligonucleotide
  source 1..34: mol_type = other DNA
ctgaacactgtggtacgaacgatgatggttgaca

SEQ ID NO: 26 - DNA, 21 bp, synthetic construct
  misc_feature 1..21: note = Synthetic primer
  source 1..21: mol_type = other DNA
ccaggggctgtgcagaatgga

SEQ ID NO: 27 - DNA, 27 bp, synthetic construct
  misc_feature 1..27: note = Synthetic primer
  source 1..27: mol_type = other DNA
tctaaaaatagagcatgaaatggctcc

SEQ ID NO: 28 - DNA, 28 bp, synthetic construct
  misc_feature 1..28: note = Synthetic primer
  source 1..28: mol_type = other DNA
gaggtcatgtgcatttaaagttgggttc

SEQ ID NO: 29 - DNA, 13 bp, synthetic construct
  misc_feature 1..13: note = Synthetic primer
  source 1..13: mol_type = other DNA
gcacgcccccggc

SEQ ID NO: 30 - DNA, 18 bp, synthetic construct
  misc_feature 1..18: note = Synthetic primer
  source 1..18: mol_type = other DNA
ggcagctctggagggcgc

SEQ ID NO: 31 - DNA, 24 bp, synthetic construct
  misc_feature 1..24: note = Synthetic primer
  source 1..24: mol_type = other DNA
actaacctgttaacttcgagctga

SEQ ID NO: 32 - DNA, 21 bp, synthetic construct
  misc_feature 1..21: note = Synthetic primer
  source 1..21: mol_type = other DNA
cctgaggtgggcactggactg

SEQ ID NO: 33 - DNA, 28 bp, synthetic construct
  misc_feature 1..28: note = Synthetic primer
  source 1..28: mol_type = other DNA
gtatggttttgcttaatttccctaacac

SEQ ID NO: 34 - DNA, 27 bp, synthetic construct
  misc_feature 1..27: note = Synthetic primer
  source 1..27: mol_type = other DNA
cttcagcacaaactccttctaagcaga

SEQ ID NO: 35 - DNA, 24 bp, synthetic construct
  misc_feature 1..24: note = Synthetic primer
  source 1..24: mol_type = other DNA
gttcgcagggtttcacaaaaatag

SEQ ID NO: 36 - DNA, 26 bp, synthetic construct
  misc_feature 1..26: note = Synthetic primer
  source 1..26: mol_type = other DNA
gagccctacttttgaatccccaggta

SEQ ID NO: 37 - DNA, 35 bp, synthetic construct
  misc_feature 1..35: note = Synthetic primer
  source 1..35: mol_type = other DNA
ctgaagcatcttagtgcttttaaattatagaatta

SEQ ID NO: 38 - DNA, 38 bp, synthetic construct
  misc_feature 1..38: note = Synthetic primer
  source 1..38: mol_type = other DNA
accacttaagatttgatttttatcattaattgtgatgc

SEQ ID NO: 39 - DNA, 27 bp, synthetic construct
  misc_feature 1..27: note = Synthetic primer
  source 1..27: mol_type = other DNA
gtatcagagccaagttcaataagcatt

SEQ ID NO: 40 - DNA, 27 bp, synthetic construct
  misc_feature 1..27: note = Synthetic primer
  source 1..27: mol_type = other DNA
ggaaatgaggttgggaccaaaagaaga

SEQ ID NO: 41 - DNA, 20 bp, synthetic construct
  misc_feature 1..20: note = Synthetic primer
  source 1..20: mol_type = other DNA
atcacctcccccagatcact

SEQ ID NO: 42 - DNA, 24 bp, synthetic construct
  misc_feature 1..24: note = Synthetic primer
  source 1..24: mol_type = other DNA
actgcctgcttgtcaacagctatc

SEQ ID NO: 43 - DNA, 26 bp, synthetic construct
  misc_feature 1..26: note = Synthetic primer
  source 1..26: mol_type = other DNA
acttccatgttttaaagcatctacgg

SEQ ID NO: 44 - DNA, 69 bp, synthetic construct
  misc_feature 1..69: note = Synthetic oligonucleotide
  source 1..69: mol_type = other DNA
tgccacagccagaaatcagcgcacggcgcgatgctgaatacttgtgataatacacctctacgggtcagg

SEQ ID NO: 45 - DNA, 74 bp, synthetic construct
  misc_feature 1..74: note = Synthetic oligonucleotide
  source 1..74: mol_type = other DNA
gagtctcttaggaccccagggttctaaggcccgcgatgctgaatacttgtgataatacacctctacgggtcagg

SEQ ID NO: 46 - DNA, 69 bp, synthetic construct
  misc_feature 1..69: note = Synthetic oligonucleotide
  source 1..69: mol_type = other DNA
ggtgggaaggagggagcacgggcttccgcgatgctgaatacttgtgataatacacctctacgggtcagg

SEQ ID NO: 47 - DNA, 76 bp, synthetic construct
  misc_feature 1..76: note = Synthetic oligonucleotide
  source 1..76: mol_type = other DNA
cagggttttctctgattcgtgtgcttctctcccagcgatgctgaatacttgtgataatacacctctacgggtcagg

SEQ ID NO: 48 - DNA, 72 bp, synthetic construct
  misc_feature 1..72: note = Synthetic oligonucleotide
  source 1..72: mol_type = other DNA
cctccccacaagtgtatgagggtggctgctgcgatgctgaatacttgtgataatacacctctacgggtcagg

SEQ ID NO: 49 - DNA, 79 bp, synthetic construct
  misc_feature 1..79: note = Synthetic oligonucleotide
  source 1..79: mol_type = other DNA
gcaaggaataacccctgtcaaaccatcagaaagaagcgcgatgctgaatacttgtgataatacacctctacgggtcagg

SEQ ID NO: 50 - DNA, 91 bp, synthetic construct
  misc_feature 1..91: note = Synthetic oligonucleotide
  source 1..91: mol_type = other DNA
cagaaattgtactagatgctgaggactaaagatacttaagtatcttatcgcgatgctgaatacttgtgataatacacctctacgggtcagg

SEQ ID NO: 51 - DNA, 79 bp, synthetic construct
  misc_feature 1..79: note = Synthetic oligonucleotide
  source 1..79: mol_type = other DNA
tcttccctcttgctttgtcaatcagttctcagtagcagcgatgctgaatacttgtgataatacacctctacgggtcagg

SEQ ID NO: 52 - DNA, 76 bp, synthetic construct
  misc_feature 1..76: note = Synthetic oligonucleotide
  source 1..76: mol_type = other DNA
gggctcaaaagatggccagcagaagaagcatagtgcgatgctgaatacttgtgataatacacctctacgggtcagg

SEQ ID NO: 53 - DNA, 78 bp, synthetic construct
  misc_feature 1..78: note = Synthetic oligonucleotide
  source 1..78: mol_type = other DNA
aaaacaaggcagtcacaaaggctagcactaccattggcgatgctgaatacttgtgataatacacctctacgggtcagg

SEQ ID NO: 54 - DNA, 77 bp, synthetic construct
  misc_feature 1..77: note = Synthetic oligonucleotide
  source 1..77: mol_type = other DNA
aatgtctagcccatagtggatgccctgttagtggtgcgatgctgaatacttgtgataatacacctctacgggtcagg

SEQ ID NO: 55 - DNA, 43 bp, synthetic construct
  misc_feature 1..43: note = Synthetic oligonucleotide
  source 1..43: mol_type = other DNA
gccgtgcgctgatttctggctgtggcagaagagatgcctacaa

SEQ ID NO: 56 - DNA, 46 bp, synthetic construct
  misc_feature 1..46: note = Synthetic oligonucleotide
  source 1..46: mol_type = other DNA
gggccttagaaccctggggtcctaagagactcatcctgaagccaag

SEQ ID NO: 57 - DNA, 41 bp, synthetic construct
  misc_feature 1..41: note = Synthetic oligonucleotide
  source 1..41: mol_type = other DNA
ggaagcccgtgctccctccttcccaccacacatggcacaaa

SEQ ID NO: 58 - DNA, 45 bp, synthetic construct
  misc_feature 1..45: note = Synthetic oligonucleotide
  source 1..45: mol_type = other DNA
tgggagagaagcacacgaatcagagaaaaccctgcagggacagca

SEQ ID NO: 59 - DNA, 41 bp, synthetic construct
  misc_feature 1..41: note = Synthetic oligonucleotide
  source 1..41: mol_type = other DNA
agcagccaccctcatacacttgtggggagggagcctccagc

SEQ ID NO: 60 - DNA, 55 bp, synthetic construct
  misc_feature 1..55: note = Synthetic oligonucleotide
  source 1..55: mol_type = other DNA
gcttctttctgatggtttgacaggggttattccttgcaaacaaaacagaccaaaa

SEQ ID NO: 61 - DNA, 65 bp, synthetic construct
  misc_feature 1..65: note = Synthetic oligonucleotide
  source 1..65: mol_type = other DNA
gataagatacttaagtatctttagtcctcagcatctagtacaatttctggttctttgtatgcagt

SEQ ID NO: 62 - DNA, 50 bp, synthetic construct
  misc_feature 1..50: note = Synthetic oligonucleotide
  source 1..50: mol_type = other DNA
tgctactgagaactgattgacaaagcaagagggaagacctttctgaggcc

SEQ ID NO: 63 - DNA, 46 bp, synthetic construct
  misc_feature 1..46: note = Synthetic oligonucleotide
  source 1..46: mol_type = other DNA
actatgcttcttctgctggccatcttttgagcccacaggcagggaa

SEQ ID NO: 64 - DNA, 47 bp, synthetic construct
  misc_feature 1..47: note = Synthetic oligonucleotide
  source 1..47: mol_type = other DNA
caatggtagtgctagcctttgtgactgccttgttttgggctgccatg

SEQ ID NO: 65 - DNA, 50 bp, synthetic construct
  misc_feature 1..50: note = Synthetic oligonucleotide
  source 1..50: mol_type = other DNA
accactaacagggcatccactatgggctagacattgtcctaagcacttca

SEQ ID NO: 66 - DNA, 47 bp, synthetic construct; identical to SEQ ID NO: 16
  misc_feature 1..47: note = Synthetic oligonucleotide
  source 1..47: mol_type = other DNA